SEQUENCE LISTING (11 sequences)

SEQ ID NO: 1 - DNA, 2607 bp, Mycoplasma suis
tacaacatttttagaagttatactgctgggatctccctaacttctaaagtagttggagagatcctcaaaggatctctcaatttattaggtagttacggaaaagttataggttcagaattatttctagttaattttgcagcctcaaaatgaaggctgcttttgactagaagagaaataaatcaaatttcagatttagttaaaagagaaaaatacgttattattccgggaggtctttttcttcagaatagaaagataaaagttgaattaaatctttgtaaatacaacagaaattatgagaaagagacttcaaagaaatataagagaaataaaagaaataaatattcagatgacgaatattaatttctttgatttatttcgataatgctgctacttctcaaaaatttccatttttttgagaagaatgactaaaaagctatattgattcttcctgaatacataaaaaacatttacacaaacaatctttactagataagttatctaaatatttgggattcaaatcccaaaatattttcttaactccctcttctacttatgcaataaatgaaatatgagaatacctacttcaagaaaagaaagatgttttgaacatctatcttttcagtagggatcatatttcaaatattgcaagtattatttataagtatcaactgaattctgaagccatcagaattcattttttagaacacaataaagagaattatgtgttattgcctaattcaattattcttctgactgtgaaagacaacttaggtatctttcacataaaagaagaaataattagaagaattaggcaggataatcctttctcattcattattggcgactttaatcaatatatgtctaattcttcagaagctgaaaaaatattcagtctatttgactgcatatatttttcagcccataaatgatttggtccttttggacttgcagtaataggctttaatagaccttcaaaaattaaatttttggaagaacctaattatttcttagattgaaggtctatttttgcctgagataaagtattttggaaaattcaggaggaaataaataagaatagaaagaaatttcgtgaattaagaaaaacatgaatagagaattttccgaaaattcctaatttaagttatcaaagttatgagaattccctcatattcttagttagatataaatctgaattctttcatgactttattttttgattggaagaaaataaagtcattttcagagctggagatttatgtagcacatatgatgatagagagttgggcttttcagccagattttctctatcaatattgaatacagtagatgagatcaaaaaattttgtgaacttgtaaaatcttttatgttacaagtaagttaaaatcctaaagtagtatgacaatccacaaagtagcaatcaatggattcggaagaatcggaagattactatttagaaatcttctttcttctcaaggagttcaagttgtagctgttaatgacgtagttgacattaaagttcttactcaccttttggtttatgacagtgctcaaggaaaactaaaagattgagaagtaagttgtgattcagaatacataagactaaagaatgtaaatactggagaagttagagaagttagagttttcaacttcaatactgaaaagatttatcactgaggtgaattagaaattgattgtgttgttgaatgttcaggaagattcttaactaaggaagcagttaagtgtcaccttgatgcaggagctcaaaaagttcttatttcagctcctgcaaaggatgacactaagacagttgtttacaacgtaaaccatactcaaattaccagctcagacaatgttatttcaggagcttcatgtacaactaatgcactagctcctatcgtaaaaattattcacagaaaatttggaattaattctggattcatgacaacagttcacgctttcacttctgaccaaagacttcaagactctcctcactctgacctaagaagagctagagcagctgctggatcaattattcctacaactacaggagcagctgctgcaattggaagagtaattccagaattgaatggaaaacttgatggaattgcacacagagtgcctgtattaactggttctctagttgacctatgtttaaaaataaataagtctgtttctgcagaagaaatcaatgaagcaattaaggatggagaaaatgaaacccttgcttatgtagaagatccaattgtatctgctgacattatcggagatacacatggttctgtttttgactcatctctaactaaagtattgccaactggagaagttaagctgtatgcatgatatgataatgagtcttcttatgtaaatcaacttgcaagaactttgaaatactacatttctctttaatttctcaataatttagattgagatttaataagctaaagctacgtgatctcaatctctcaaataaaagagttgtacttagattagacttaaacgttcccgttaaagacgggaaaattctaaataatacaagactattaggtactattgaaactattcaatacctactagaaaacaagtgtagtgttgttatattaagtcac

SEQ ID NO: 2 - protein, 336 aa, Mycoplasma suis
MTIHKVAINGFGRIGRLLFRNLLSSQGVQVVAVNDVVDIKVLTHLLVYDSAQGKLKDWEVSCDSEYIRLKNVNTGEVREVRVFNFNTEKIYHWGELEIDCVVECSGRFLTKEAVKCHLDAGAQKVLISAPAKDDTKTVVYNVNHTQITSSDNVISGASCTTNALAPIVKIIHRKFGINSGFMTTVHAFTSDQRLQDSPHSDLRRARAAAGSIIPTTTGAAAAIGRVIPELNGKLDGIAHRVPVLTGSLVDLCLKINKSVSAEEINEAIKDGENETLAYVEDPIVSADIIGDTHGSVFDSSLTKVLPTGEVKLYAWYDNESSYVNQLARTLKYYISL

SEQ ID NO: 3 - DNA, 4350 bp, Mycoplasma suis
gaggatgtttttcaattaactttctaattcgttgaaagtcttttctcttattcagctctctatatatgaactcttcaaaagtttgagtcccttcggactctttaaagcccttaacataactagacattgaatgatatagagaacagaatacaaatgggaaagatagctgggaaataattttcccagcaaacatagaagaaccttctattagagaaattactaatataggagtttgttcttcttcagagatatcattctgggaataatatcagttaagtctttgagctaagaagtagataatattttctacttcatatttattaagtaattgtttagagaaagattttgtttgaattttctttccaaagatttgactataaatctttggaagctcccttaaatgtctctctagattgaaacaatcttctcaatctttattggaaagaagtttctttataggagaagattgaatagcttctcataaagatgggaagttcccatcttttacttctttagaaagagaagaaagaagtgtatgagcttcttctcttgtacttatagaagaagattctataagttttaggagaatactttgagaagatataagcccattagttaactgaaggtttttagaaataccttcagaattggcttttatcttcttaaggaatttgtgcattctagtaacaatattgaatgcgagaataggagcatccattaagctcattctctcattagaagagtgactaatatctctttcttctcacaaaaagttattgtctttagctagagaagaaagactatctagtcacttagttaatccagtaatgttttccaactctactggattaagtttgtgcggcatagaagaagagcccacactatctttaggtttaagtatttctatttcatttatctcttctctcataaaggttcttagagtaagagctaaagagttaataatagaacctatatgagagagggaataaatataagaagaatatctatttctaggtagagcttgtgtagagcctgaaatagtatatagtccaaatctagaagataattcttcttggacttcaggtccaatatgtgcatatgtccctgtagatcccttaatactaattacttctaaatatctcctagatatataaagactttcaagagctgactctaattcttgatatgttattgcgaacctatatccaaaagatgtaggttctgcatgtctaccatgtgttctacctacttgaataaagtctctatattcattagctaatttatatagagtgtcttgcagactctctatttcttttataagtaatttattagcttctctaagagctaatgaattagagctatctatgatatctgaactagtaattccataatgtatatatttagaagctgggtttctacccagctttctttctagtaatctaaggaaagctacgaagtcgtgctgagttttcatttcctcagctactacttcatagcttggaatttctggtcattctagttctagttttgatatctccttgtcggagatagaaaatcttctagctagagaatatagtatttctttctcaagaatagatcatcttttatatttggaattttctgaaaaaatatgttcaagttgtgggacttcatatctcttgatcattcgtattttatatttctaattctataaggaatttggttagatatcccaacaggataagggccattagaaatattttgggcataatttttagcagtttaaaaaaattatgctaaaataattttcgaatggtagcaaagaaagaaataattctaggtattgacctaggtacaactaactcctgtgtagctgtaatagaatcaggagctcctaaggtattagaaactccagaaggaaagagaacagttccttctgtagtttcctttaaaggtaaagaaataatagtaggagatagtgctaagagacaaatggttacaaataagaacactatcttctctattaagagactaattggaactgatcaaaaagtaactgctcaaggaaaagaatatagtccagaagaaatttctgcatatattcttgcctacataaaagaatatgcagaaaagagagttggagaaaaagtacaaaaagctgttattactgttccagcatactttaatgactctcaaagacagtcaacaaagaatgctggaaagattgctggactagaagtagttcgaattgttaatgaacctacagcagctgcgctagcttacggactagataaaaaagaagaagaaaagaagatacttgtttatgacctagggggaggtacttttgatgtttcccttctggaagtttccgatggaactttccaagttttggcaacatcaggagataacaatctaggtggtgatgactgagaccaaagaattattaagtgacttttggaatcaatccaaaaggaacatagtgtagatctatctaaagataacttagtaatgcaaagattaaaagaagctgctgaaaaggcaaagattgaactttcttcagttcaacaaacacaaattatgcttcctttcctttcaatggttcgtggagaaccattgaatgttgatttctcattaactagagaacaattccaactatttactaaggacttactagaaagaacaatagcacctgttaaggatgctattgcagaatctaaattatcactttcagatataaatgaagttctactagtaggtggttctactagaatgcctgcagtacaagaactagtagaaaaattaactggaaagaaacctaacttgtctattaacccagatgaagtagtagctctaggagcttctgttcaagctggaattctagcaggagatattaaagatattctccttctagacgtaacacctctaactctaagtattgaaacactaggtggagttgctactcctctaattcctagaaatagtactattccaattgataagaagcaattgttctcaactgctgtagacaatcaacctagtgttgatattcacgtagtacaaggtgaaagacctatggctaaccaaaacaaatccctaggtactttcacccttcaaggaattaagcaagctcctaagggaatgccaaaaatagaagtatccttctctattgacgctaacggtattcttaccgttaaagcagaagataaggatactggaaagcaaaacaatataactattaatcaagcttctggattatcagaagaagaaattaataagataatccgagaagctgaagaaaatcttgaacaagataagaaggttaaggaagaaatagaaattaagaatgaagctgaatcttggatttctatgctagaaaaccaaatgaaggatgattcttcaaagattcctgaagcaagtatagaagaaactaagaagttaattgaagaatttaagaaacttcttgaagaaaagaagtatgatgaacttaaggctaagatgaatcaactaaaagaaatgagtcaaaaaatgatgcaagaagtttatcaacaacaacaagctgctggaggacaagctgcatcagaagaaaaaggtcctgaaggagaagacatcaaagaagtagaacttaatgaagaaagtaattagtttcaaagttaagaactaaatatttagtcttttttctgaggagtttttactcctcagtcttttaaattaaaactgtattgttaatttatttagttaattaatttgagttccgaagaaagaaagtctaacttaaaggattcagagttagactctatagttctaaaactactatctgttgatagtagaccagtaccttttaatgtattgaagtccaaattctttagatttataagagctaatcatcctagagaatttgttcttgaagaatctttcttcaatacattaacaagactaaagaaaaaataccttataggtgaaaaccaatatggtaagtggtttatagactatctagactataaacacactaatagatttggagaaggcttcttagatatagaaccaagaagtggtaatggattcattacagtaaagaaagaaggagaaaaatataagaagtcttctcactttgtgcacaagaaaaatctcaatggagctaagactggagatttagtaaaatttgttgaattggagataaatctcaagagaaaagcagctagattttctcttatagatgcttctgtagtagaagttctgagtcattcttcaaaagactcagaagtatctaataatgagtaatagtaacgaaaaaataattcaatcagcttcccaaaagactgaacttctgttcagaaatcctaattctagacctactgagcaagaacttgctag

SEQ ID NO: 4 - protein, 609 aa, Mycoplasma suis
MVAKKEIILGIDLGTTNSCVAVIESGAPKVLETPEGKRTVPSVVSFKGKEIIVGDSAKRQMVTNKNTIFSIKRLIGTDQKVTAQGKEYSPEEISAYILAYIKEYAEKRVGEKVQKAVITVPAYFNDSQRQSTKNAGKIAGLEVVRIVNEPTAAALAYGLDKKEEEKKILVYDLGGGTFDVSLLEVSDGTFQVLATSGDNNLGGDDWDQRIIKWLLESIQKEHSVDLSKDNLVMQRLKEAAEKAKIELSSVQQTQIMLPFLSMVRGEPLNVDFSLTREQFQLFTKDLLERTIAPVKDAIAESKLSLSDINEVLLVGGSTRMPAVQELVEKLTGKKPNLSINPDEVVALGASVQAGILAGDIKDILLLDVTPLTLSIETLGGVATPLIPRNSTIPIDKKQLFSTAVDNQPSVDIHVVQGERPMANQNKSLGTFTLQGIKQAPKGMPKIEVSFSIDANGILTVKAEDKDTGKQNNITINQASGLSEEEINKIIREAEENLEQDKKVKEEIEIKNEAESWISMLENQMKDDSSKIPEASIEETKKLIEEFKKLLEEKKYDELKAKMNQLKEMSQKMMQEVYQQQQAAGGQAASEEKGPEGEDIKEVELNEESN

SEQ ID NO: 5 - protein, 12 aa, Mycoplasma suis
EELESNLGTIAK

SEQ ID NO: 6 - protein, 16 aa, Mycoplasma suis
SGKYDLDFKSPDDPSR

SEQ ID NO: 7 - protein, 18 aa, Mycoplasma suis
VAPEEHPVLLTEAPLNPL

SEQ ID NO: 8 - DNA, 24 bp, Mycoplasma suis
acaactaatgcactagctcctatc

SEQ ID NO: 9 - DNA, 26 bp, Mycoplasma suis
gctcctgtagttgtaggaataattga

SEQ ID NO: 10 - DNA, 32 bp, Mycoplasma suis
caagactctcctcactctgacctaagaagagc

SEQ ID NO: 11 - DNA, 27 bp, Mycoplasma suis
ttcacgctttcacttctgaccaaagac

(19) United States
(12) Patent Application Publication
(10) Pub. No.: US 2009/0068218 A1
(43) Pub. Date: Mar. 12, 2009

(54) Antigens for Vaccination Against and Detection of Mycoplasma Suis

(75) Inventors: Katharina Hoelzle, Zurich (CH); Ludwig E. Hoelzle, Zurich (CH); Max M. Wittenbrink, Jonen (CH)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UNIVERSITAT ZURICH, Zurich (CH)

(21) Appl. No.: 12/065,986

(22) PCT Filed: Aug. 8, 2006
(86) PCT No.: PCT/CH2006/000417

§ 371 Date: Sep. 29, 2008

(30) Foreign Application Priority Data
Sep. 9, 2005 (EP) 05019620.3

(51) Int. Cl. A61K 39/02; C07K 14/30; C12N 15/31; C07H 21/04; C07K 16/12; G01N 33/569; C12Q 1/68

(52) U.S. Cl. 424/190.1; 530/350; 536/23.7; 536/24.5; 530/387.9; 435/7.1; 435/7.92; 435/6

(57) ABSTRACT

The present invention relates to antigens for vaccination against and detection of Mycoplasma suis (M. suis) and related haemotrophic Mycoplasma species. Furthermore, the present invention relates to polynucleotides encoding such antigens, vectors containing the polynucleotides, host cells comprising the polynucleotides and/or vectors as well as methods for the treatment of infections by and vaccination against M. suis and related pathogens.

This application is a national phase application under 35 U.S.C. § 371 of International Application WO 2007/028259, filed on Aug. 8, 2006, which claims the benefit of EP Application 05019620.3, which was filed on Sep. 9, 2005, both of which are incorporated herein by reference in their entirety.

The present invention relates to antigens for vaccination against and detection of Mycoplasma suis (M. suis) and related haemotrophic Mycoplasma species. Furthermore, the present invention relates to polynucleotides encoding such antigens, vectors containing the polynucleotides, host cells comprising the polynucleotides and/or vectors as well as methods for the treatment of infections by and vaccination against M. suis and related pathogens.

M. suis (formerly Eperythrozoon suis) belongs to a group of haemotrophic bacteria. M. suis is an epicellular haemoparasite that attaches to and causes deformity and damage to porcine erythrocytes. The resulting disease, traditionally called porcine eperythrozoonosis (PE), has been reported worldwide and is considered a problem of feeder pigs where it manifests as a febrile acute icteroanaemia with low morbidity and high mortality. Chronic low-grade M. suis-infections vary from asymptomatic infections to a range of clinical conditions including (i) anaemia, mild icterus, and general unthriftiness in newborns, (ii) growth retardation in feeder pigs, and (iii) poor reproductive performance in sows. Moreover, M. suis is suspected of suppressing the host's immune response leading to an increased proneness for other infectious agents of porcine respiratory and enteric diseases.

The lack of an in vitro cultivation system is the crucial barrier to systematic analyses of the biology of M. suis as well as for the development of valuable diagnostic procedures for e.g. the accurate assessment of the prevalence and significance of M. suis in pig populations. Hitherto, laboratory diagnosis of M. suis relies on the microscopic examination of chemically stained peripheral blood smears to directly visualize the microorganisms attached to erythrocytes. The drawbacks of microscopy include problems with both specificity and sensitivity, because the readily identifiable but short-term bacteraemia linked with the onset of acute disease is lacking in chronic infections.

An efficient method of control of porcine eperythrozoonosis caused by M. suis is eradication of infection by detection and removal of infected carrier animals. For these purposes serological assays are still the methods of choice. A specific and sensitive serological assay based on defined M. suis antigens would allow extensive prevalence studies and is applicable as a matter of routine in diagnostic laboratories. However, attempts to analyse the humoral immune response of pigs to M. suis were impeded by the poor sensitivities and specificities of current antibody assays which comprise the complement fixation test (CFT), the indirect hemagglutination assay (IHA), and the enzyme-linked immunosorbent assay (ELISA). Serodiagnostic assays described so far have the intrinsic disadvantage of employing complex and undefined M. suis antigens obtained from the peripheral blood of experimentally infected pigs. The only assay that could presently be considered a gold standard to examine swine herds for chronic M. suis infections is the provocation of acute disease by means of splenectomy and microscopic confirmation of bacteraemia in pigs (Heinritzi, 1984 Tierarztl. Prax. 12: 451-454).

However, splenectomy of pigs is not suitable for routine diagnosis. Recently, molecular methods such as DNA hybridisation and PCR assays have been developed to overcome problems associated with the low sensitivity in diagnosing chronic PE with a low level of bacteraemia by microscopy. But there is no standard method which can be used in routine laboratories.

M. suis is treated pharmacologically using Tetracycline. When performing this therapy, it is possible to cure the infected pigs from the clinical symptoms during the PE attack, but it is impossible to eliminate M. suis from infected pigs. Therefore, persistent and clinically inapparent infected pigs remain carrier animals which are hot spots for the transmission of M. suis within the herd or between herds.

A vaccination has not been developed because of the fact that M. suis can not be cultivated in vitro. Therefore, potential vaccine candidates would be derived from porcine blood of clinically acute ill pigs which leads to two main restrictions: i) vaccines contain components of the porcine blood which lead to considerable side effects, i.e. an immune response against alloantigens (alloimmunity) and ii) the M. suis isolates are fully virulent and there is no possibility to attenuate these isolates by e.g. culture passages. Furthermore, so far no information is available about the immunogenic structure of M. suis and the immune response of PE which could be the base for the choice of appropriate vaccine constructs.

Therefore, the technical problem underlying the present invention is the provision of novel compounds and methods for reliable diagnosis (serology, molecular) and vaccination against as well as therapy of infection by M. suis and related bacteria.

The solution to the above technical problem is provided by the embodiments of the present invention as defined in the claims.

The present invention is particularly based on the availability of huge amounts of M. suis bacteria produced in experimentally infected pigs which allowed studies with respect to the antigenic and genetic structure of M. suis. Due to this fact it was possible to perform detailed one- and two-dimensional Western Blot analyses, and to construct a genomic DNA library of M. suis. In addition, this pig model allowed the analysis of the nature and kinetics of the immune response in the use by M. suis.

Thus, according to a first aspect the present invention relates to a vaccine against infection by haemotrophic Mycoplasma species, in particular M. suis, wherein the vaccine contains at least one peptide or polypeptide comprising at least one antigenic determinant of a protein selected from M. suis proteins having an apparent molecular weight of about 33 kDa, 40 kDa, 45 kDa, 57 kDa, 61 kDa, 70 kDa, 73 kDa and 83 kDa in a continuous 12% polyacrylamide gel in 0.025 M Tris/0.192 M glycine/0.1% SDS aqueous solution and being reactive against serum from an M. suis positive animal, in particular an M. suis-infected pig.

According to a preferred embodiment, the vaccine contains at least one peptide or polypeptide comprising at least one antigenic determinant of the protein which has an apparent molecular weight of 40 kDa as determined under the experimental conditions referred to above.

Thus, the above antigen according to the present invention contains at least one epitope (antigenic determinant) of the above-defined proteins derived from M. suis as determined by standard SDS-PAGE (see Laemmli (1970) Nature 227, 689) and Western blotting using serum from one or more pigs known to be infected by M. suis (e.g. by hitherto usual methods for the detection of M. suis as described above such as splenectomy and microscopic confirmation of bacteraemia).

The source of M. suis proteins as defined above is may be any sample or specimen in which M. suis and material derived from this pathogen can be found. In particular, sources of M. suis material preferably are specimens from infected individuals, especially pigs, such as organ tissue and body fluids (e.g. spleen tissue, blood and its parts, e.g. serum, cerebrospinal fluid, synovial fluid, lymph fluid etc.). The M. suis cells may be purified from such sources as described in Hoeizle et al. (2003) Vet Microbiol. 93: 185-196.

Further purification may comprise one or more centrifugation steps. The M. suis sample may be stored until use for sodium dodecyl sulphate polyacrylamide gel electrophoresis (SDS-PAGE). For electrophoresis, the M. suis cells are conveniently lysed in a lysis buffer known to persons skilled in the art. After lysis of the M. suis cells, the lysate is subjected to SDS-PAGE according to the method described by Laemmli (1979), supra. In order to determine the apparent molecular weight of the separated proteins, an Mw standard (commercially available, e.g. from Sigma-Aldrich, Munich, Germany) is run on the same gel. After electrophoresis, the gel is subjected to Western blotting, e.g. in a semidry blotting apparatus (commercially available, for example, from Hoefer, Amersham Bioscience) in order to immobilise the separated proteins in the SDS gel onto a membrane (e.g. nitrocellulose, PVDF).

The above-defined antigenic proteins derived from M. suis are identified by incubation with sera from M. suis-infected animals and, thereafter, by use of a second antibody such as goat-anti-pig IgG labelled with a suitable marker such as horseradish peroxidase, biotin, radioactive iodine etc. Thereafter, the blots are typically compared to negative control antigens from body fluid of non-infected animals. In addition, the above-described methods for the identification of the antigenic proteins derived from M. suis may be adapted to a preparative or micro-preparative scale such that the proteins can be obtained from the SDS gels and used as antigens in a vaccine of the present invention as such or may be further purified and/or fragmented to suitable peptide fragments containing an antigenic determinant.

According to the present invention, the terms “peptide” and “polypeptide” are used synonymously, i.e. the above terms comprise any condensation product of amino acids being connected to one another by peptide bonds in an acid amide fashion. The only further essential feature of such a peptide or polypeptide is that the respective chemical entity comprises an “antigenic determinant” derived from M. suis or a related species.

As used herein, an “antigenic determinant” means a three dimensional structure on the surface of an antigen which is capable of inducing an immune response, in particular such that antibodies are produced which are capable of binding specifically to that antigenic determinant (epitope) via their antigen binding regions. An antigenic determinant may contain amino acids, carbohydrates or lipids. The antigenic determinant present on the proteins of the present invention are usually formed by at least 5, more preferred at least 7 amino acids. The antigenic determinant may be formed by amino acids being present in a continuous sequence (continuous or sequence determinant) or it may be formed by amino acids that assemble to an epitope due to the folding of the polypeptide (discontinuous or conformational determinant).

The antigenic determinant may be present on the protein or a fragment of the protein itself, or it may be coupled to a suitable haptene.

Preferred further components of the vaccine of the present invention are adjuvants which improve the immune response against the antigenic determinant. Typical adjuvants contain aluminium compounds, in particular aluminium hydroxide, and mineral oils which are applied together or without inactivated bacteria. The most well-known adjuvant is complete Freund's adjuvant which typically contains mineral oil, an emulsifier (lanoline) and a suspension of deactivated mycobacteria. Incomplete Freund's adjuvant contains no mycobacteria. Suitable vaccine adjuvants are disclosed in the prior art; see, e.g., Hackett (2003) Vaccine Adjuvants, Humana Press: Topowa, N.J.

Furthermore, the present invention provides specific sequences of antigenic determinants of proteins which are especially useful for haemotrophic Mycoplasma-specific diagnosis, detection, vaccination and therapy.

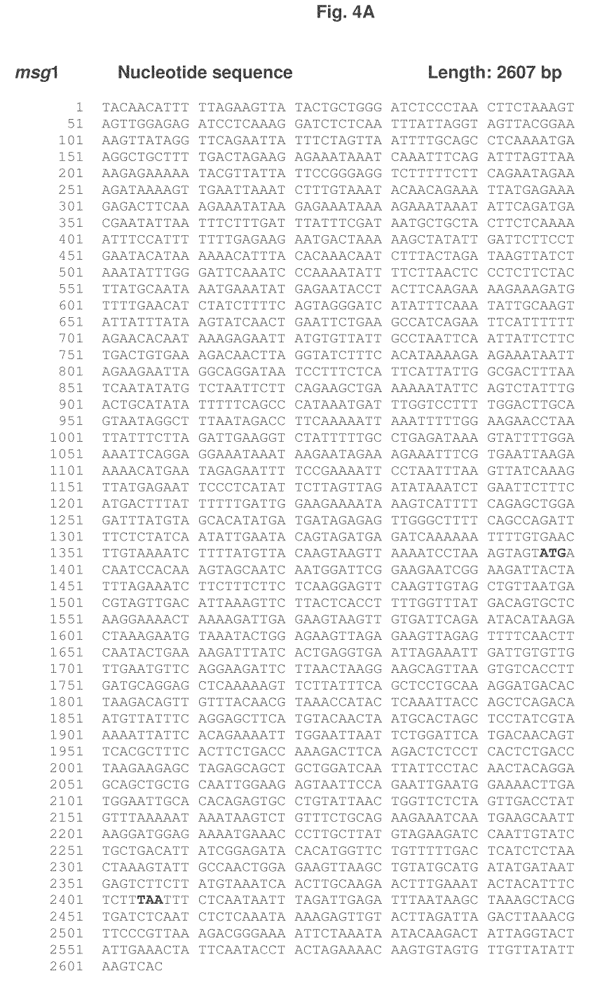

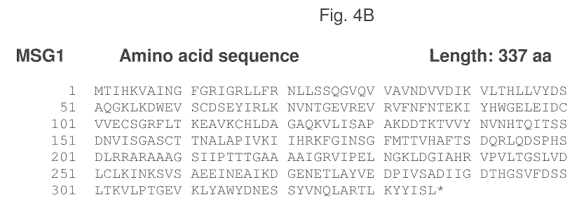

Therefore, a further aspect of the present invention is a polynucleotide comprising a sequence encoding an amino acid sequence comprising at least one antigenic determinant (epitope) of the amino acid sequence shown in FIG. 4B (SEQ ID NO: 2) or 5B (SEQ ID NO: 4).

According to a preferred embodiment the present invention relates to a polynucleotide comprising a nucleotide sequence encoding a continuous antigenic determinant contained in the sequence shown in FIG. 4B (SEQ ID NO: 2) or 5B (SEQ ID NO: 4). Preferably, the polynucleotide comprises a sequence encoding an amino acid sequence having at least 80%, preferably 90%, in particular at least 95% homology to at least 5, preferably to at least 7 consecutive amino acids of the amino acid sequence shown in FIG. 4B (SEQ ID NO: 2) or 5B (SEQ ID NO: 4).

More particularly, the polynucleotide of the present invention comprises a sequence encoding a protein having at least 80%, preferably 90%, in particular at least 95% homology to the sequence shown in FIG. 4B (SEQ ID NO: 2) or 5B (SEQ ID NO: 4), or an antigenic fragment, variant, mutant or analogue of said sequences.

The term “homology” means that the protein sequences in question have a certain percentage of their amino acid residues in common. Thus, 50% homology means that fifty of one hundred amino acids positions in the sequences are the same.

The polynucleotide according to the present invention may be a DNA, RNA or a polynucleotide comprising one or more modified nucleotides. The polynucleotide may be present in single or double-strained form. DNA, in particular double-strained DNA, forms are specially preferred. The polynucleotide of the present invention may be produced by chemical or enzymatic synthesis (cf. Gassen et al., Chemical and Enzymatic Synthesis of Gene Fragments: A Laboratory Manual, Weinheim: Verl. Chemie 1982). Preferably, polynucleotide constructs of present invention are made by recombinant gene technology (see, e.g., Sambrook et al., “Molecular Cloning”, Cold Spring Harbor Laboratory Press, New York, 1989).

An “antigenic fragment” of the polypeptide encoded by the polynucleotide of the present invention is a part or region of the complete polypeptide, in particular a fragment capable of inducing an immune response in an animal or human, especially an animal or human susceptible to infection by haemotrophic Mycoplasma species. A “variant” of the polypeptide encoded by the polynucleotide of the invention is a functional or non-functional equivalent of the original polypeptide derived from another species, in particular haemotrophic Mycoplasma species, or a functional or non-functional derivative of the original polypeptide that arises from alternative splicing or post-translational processing, but which variant retains at least the function of being an antigen as defined above with respect to the antigenic fragment.

A “mutant” of the polypeptide encoded by the polynucleotide of the invention is derived from the naturally occurring protein by insertion, substitution, addition and/or deletion of one or more amino acid residues. Amino acid substitutions may be conservative or non-conservative. Conservative amino acid substitutions are substitutions that do not substantially change the chemical character (such as size, hydrophobic/hydrophilic nature, charge, aliphatic/aromatic nature etc.) of the substituted amino acid residue. Examples of conservative amino acids substitutions are Val/Ala, Asn/Gln, Asp/Glu and Ser/Thr substitutions.

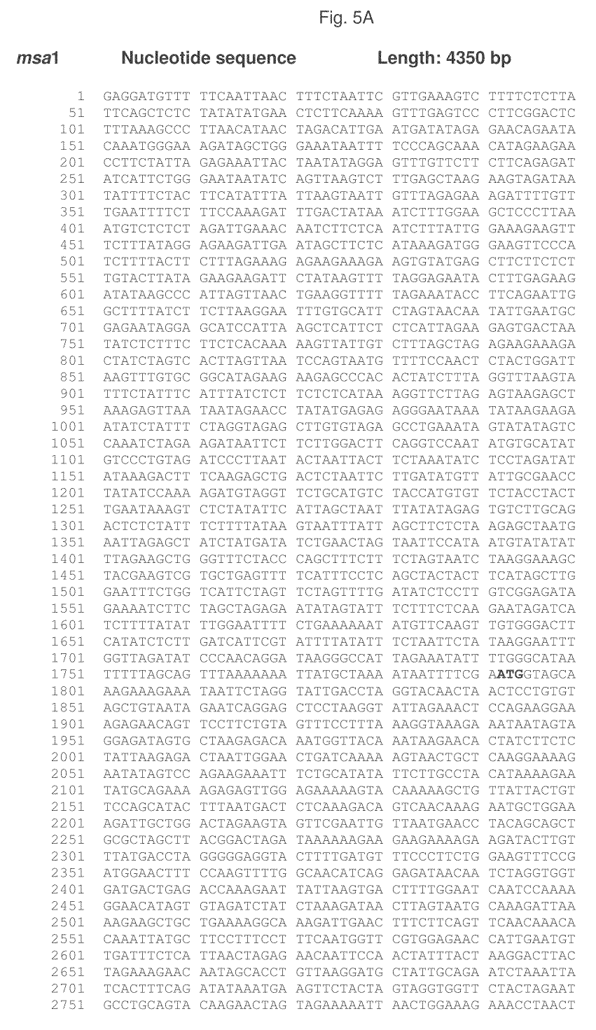

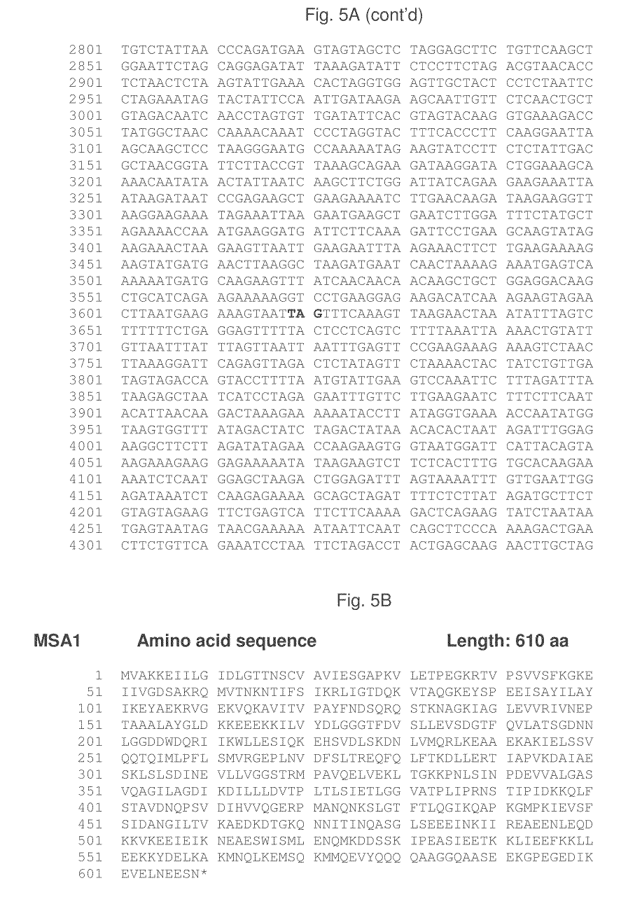

Particularly preferred polynucleotides of the present invention comprise the sequence shown in FIG. 4A (SEQ ID NO: 1), most preferred nucleotides 1397 to 2407 thereof, or FIG. 5A (SEQ ID NO: 3), most preferred nucleotides 1792 to 3621 thereof, or sequences having at least 70%, preferably at least 85%, more preferred at least 90%, in particular 95% homology to said sequences, and sequences which hybridise under standard hybridisation conditions to said sequences as well as to complementary sequences thereof.

Depending on the nucleic acid species, standard hybridisation conditions are represented by temperatures of between about 42 and about 58° C. in an aqueous buffer of between about 0.1 to 5×SSC (1×SSC=0.15 M NaCl, 15 mM sodium citrate, pH 7.2), optionally in the presence of about 50% formamide, e.g. 42° C. in 5×SSC, 50% formamide. Preferred hybridisation conditions for DNA:DNA hybrids are 0.1×SSC at temperatures of between about 20° C. to 45° C., more preferred between about 30° C. to 45° C. Preferred hybridisation conditions for DNA:RNA hybrids are 0.1×SSC at temperatures between about 30° C. to 55° C., more preferred between about 45° C. to 55° C. The hybridisation temperatures given above are examples of melting temperatures calculated for a nucleic acid having a length of about 100 nucleotides and a G+C content of 50% in the absence of formamide. Experimental conditions for DNA hybridisations are described in the prior art (see, e.g., Sambrook et al. “Molecular Cloning”, Cold Spring Harbor Laboratory, 1989) and a person skilled in the art is able to calculate individual conditions in dependence of the length of the nucleic acids, the type of hybrids and the G+C content. Further information about nucleic acid hybridisations is provided by the following references: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

The polynucleotide of the present invention comprises fragments, variants, mutants and analogues of the sequences shown in FIG. 4A (SEQ ID NO: 1) and 5A (SEQ ID NO: 3), in particular fragments, variants, mutants and analogues of the sequence of nucleotides 1397 to 2407 shown in FIG. 4A (SEQ ID NO: 1) or nucleotides 1792 to 3621 shown in FIG. 5A (SEQ ID NO: 3). A “fragment” of the above sequences is a part or region of the original sequence. A “variant” is a sequence found in a different species compared to the original sequence, or it may encode a splicing variant or post-translationally processed version of a polypeptide the original nucleotide sequence codes for. Specific variants of the polynucleotide according to the invention are found in haemotrophic Mycoplasma species other than M. suis such as M. wenyonii, M. haemofelis and M. haemocanis.

A “mutant” of the polynucleotide is derived from the parent polynucleotide by insertion, substitution, addition, inversion and/or deletion of one or more nucleotides. Specific mutants of the sequences shown in FIG. 4A (SEQ ID NO: 1) and 5A (SEQ ID NO: 3) are derived by alternative codon usage compared to the codon usage found in haemotrophic Mycoplasma species. Particular preferred mutants are designed to use the codon usage of suitable host cells such as E. coli for the production of corresponding polypeptides. In particular, TGA encodes Trp instead of a stop codon in standard codon usage; see translation Table 4 of the NCBI taxonomy database; Benson et al. (2000) Nucleic Acids Res. 28: 15-18; Wheeler et al. (2000) Nucleic Acids Res. 28:10-14).

The “analogue” of the polynucleotide encodes a functional equivalent of the polynucleotide but containing one or more non-naturally occurring nucleotides. The modification of the analogue in comparison to the natural nucleotide may occur at the base as well as at the sugar and/or phosphoric acid moiety of the nucleic acid building block. Specific examples of nucleotide analogues are phosphoroamidates, phosphorothioate, peptide nucleotides (i.e. the polynucleotide is at least in part characterised by a backbone of peptide bonds, thus representing a PNA), methyl phosphonate, 7-deazaguaonsine, 5-methylcytosine and inosine.

The present invention is also directed to nucleotide sequences capable of controlling the expression of the above-defined polynucleotides encoding the polypeptides of the invention. Such control sequences are derived from the genes which comprise the coding sequences for the polypeptides of the invention. Preferred nucleotide sequences comprise nucleotides 1 to 1396 and/or nucleotides 2408 to 2607 shown in FIG. 4A (SEQ ID NO: 1) and/or nucleotides 1 to 1791 and/or nucleotides 3622 to 4350 shown in FIG. 5A (SEQ ID NO 3), or a functionally active fragment, variant, mutant or analogue of said sequences.

Preferred sequences for controlling the expression of a polypeptide having the amino acid sequence shown in FIG. 4B (SEQ ID NO: 2), or a polypeptide derived from said amino acid sequence, are derived from nucleotides 1 to 1396 and/or nucleotides 2408 to 2607 shown in FIG. 4A (SEQ ID NO: 1). Preferred sequences for controlling the expression of a polypeptide having the amino acid sequence shown in FIG. 5B (SEQ ID NO: 4), or a polypeptide derived from said amino acid sequence, are derived from nucleotides 1 to 1791 and/or nucleotides 3622 to 4350 shown in FIG. 5A (SEQ ID NO 3).

A further embodiment of the present invention is an antisense nucleic acid directed against the above-defined polynucleotide.

An antisense nucleic acid has a nucleotide sequence which is at least in part complementary to the target sequence The antisense nucleic acid of the present invention is a single or double-strained nucleic acid which is at least in part complementary to at least 8, preferably at least 10 consecutive nucleotides of the sequences of nucleotides 1397 to 2407 shown in FIG. 4A (SEQ ID NO: 1) or nucleotides 1792 to 3621 shown in FIG. 5A (SEQ ID NO: 3). Preferred antisense nucleic acids according to the present invention are molecules which are capable of binding to a polynucleotide having the full or a partial sequence of nucleotides 1397 to 2407 shown in FIG. 4A (SEQ ID NO: 1) or nucleotides 1792 to 3621 shown in FIG. 5A (SEQ ID NO: 3).

According to the present invention, the term “antisense nucleic acid” comprises also peptidic nucleic acids (PNA) which are characterised by a peptide backbone linking the nucleobases. Further preferred antisense nucleic acids for use in the present invention are part of catalytic nucleic acids such as ribozymes, in particular hammerhead ribozymes, or DNA enzymes, in particular of the type 10-23. A ribozyme is a catalytically active RNA, a DNA enzyme a catalytically active DNA.

Useful antisense nucleic acids in the context of the present invention are typically DNA or RNA species containing or consisting of unmodified or modified nucleotides. Especially in the case of antisense RNA molecules, it is preferred to incorporate at least one analogue of naturally occurring nucleotides in order to increase the resistance against degradation by RNAses. This is due to the fact that the RNA-degrading enzymes of cells preferably recognise naturally occurring nucleotides. Therefore, the degradation of the RNA can successfully be diminished by incorporating nucleotide analogues into the RNA.

As already mentioned with respect to analogues of the polynucleotide according to the present invention, the modification of the analogue in comparison to the natural nucleotide may occur at the base as well as at the sugar and/or phosphoric acid moiety of the nucleic acid building block. Specific examples of nucleotide analogues are mentioned above.

According to a preferred embodiment antisense nucleic acids of the present invention are capable of inhibiting the expression of the polynucleotide of the present invention substantially, for example by at least 80%, preferably at least 90%, more preferred at least 95%, or even more in comparison to the normal or naturally occurring expression level found in haemotrophic Mycoplasma species, in particular M. suis.

Furthermore, the present invention relates to a vector containing the polynucleotide and/or the antisense nucleic acid as defined above.

The vector according to the present invention is a linear or circular nucleic acid molecule which is preferably derived from plasmids, virus, phages or cosmids or other artificial nucleic acids constructs being capable of introducing and amplifying/replicating the polynucleotide or antisense nucleic acid in a suitable host. Vectors of the present invention are preferably capable of autonomous replication in the host. Thus, the vector contains typical components such as at least one origin of replication (Ori), one or more unique restriction sites (MCS, multiple cloning site(s)) one or more marker genes such as antibiotic resistance markers, for example against kanamycin, ampicillin, gentamicin, chloramphenicol etc. for selection of successfully transformed host cells. Especially preferred vectors of the present invention are expression vectors which preferably contain a suitable promoter, operator and terminator sequences for transcription and sequences for ribosomal entry sites in order to start translation of the corresponding mRNA.

Thus, according to a preferred embodiment, the vector of the present invention contains at least one promoter sequence operatively linked to the polynucleotide and/or antisense sequence, thus capable of controlling the expression of said polynucleotide/antisense nucleic acid. Suitable promoters in constructs of the present invention are e.g. common bacterial promoters such as the lac promoter and derivatives thereof, e.g. tac, which are inducible by addition IPTG. Other preferred inducible bacterial promoters are AraC/pBAD systems. Furthermore, the vector of present invention may contain phage promoters for expression in bacterial systems. Preferred examples of phage promoters are the T7, lambda P_{L} and SP6 promoters. Further preferred elements that may be present in the vector of the present invention are sequences for termination of transcription (terminator sequences), and sequences regulating the expression of the polynucleotide and/or antisense nucleic acid such as enhancer and/or repressor sequences. Vectors of the present invention preferably contain control sequences derived from the genes encoding the polypeptides of the present invention. Especially preferred control sequences are define above.

Especially preferred vectors according to the present invention are bacterial expression vectors wherein the polynucleotide can be cloned in frame to one or more sequences coding for peptides/polypeptides serving as markers or tags for facilitating the detection and/or purification of the construct. Such tags or markers may be present N- and/or C-terminally on the expressed polypeptide. Typical examples are sequences coding for His tags, GST (glutathione S transferase), proteins providing fluorescence markers such as GFP, YFP etc.

A further aspect of the present invention is a host cell containing the polynucleotide and/or the antisense nucleic acid and/or the vector of the present invention. Typically, the host cell will be selected according to the vector (if such a vehicle is used) chosen for the propagation/expression of the polynucleotide/antisense nucleic acid.

Preferred host cells are selected from procaryotic hosts such as bacteria, in particular E. coli and haemotrophic Mycoplasma species, in particular M. suis, M. wenyonii, M. haemofelis and M. haemocanis. Other useful host cells eukaryotic host cells, e.g. yeast cells such as S. cerevisiae, P. pastoris etc.

Furthermore, the present invention is directed to polypeptides encoded by the polynucleotide as defined above. Thus, the polypeptide according to the present invention contains at least one antigenic determinant of MSG1 (amino acid sequence according to FIG. 4B (SEQ ID NO: 2)) or MSA1 (amino acid sequence according to FIG. 5B (SEQ ID NO: 4)). Preferred embodiments of the polypeptide according to the present invention comprise amino acid sequences shown in FIG. 4B (SEQ ID NO: 2) or 5B (SEQ ID NO: 4) or amino acid sequences which contain antigenic fragments, variants, derivatives or mutants of said sequences.

The present invention also relates to an antibody directed against the above-defined polypeptide. The term “antibody” comprises polyclonal as well as monoclonal antibodies, chimeric antibodies, genetically engineered, e.g. humanised, antibodies, which may be present in bound or soluble form. Furthermore, an “antibody” according to the present invention may be a fragment or derivative of the afore-mentioned species. Such antibodies or antibody fragments may also be present as recombinant molecules, e.g. as fusion proteins with other (proteinaceous) components. Antibody fragments are typically produced by enzymatic digestion, protein synthesis or by recombinant technologies known to a person skilled in the art. Therefore, antibodies for use in the present invention may be polyclonal, monoclonal, human or humanised or recombinant antibodies or fragments thereof as well as single chain antibodies, e.g. scFv-constructs, or synthetic antibodies.

Polyclonal antibodies are heterogenous mixtures of antibody molecules being produced from sera of animals which have been immunised with the antigen. Subject of the present invention are also polyclonal monospecific antibodies which are obtained by purification of the antibody mixture (e.g. via chromatography over a column carrying peptides of the specific epitope). A monoclonal antibody represents a homogenous population of antibodies specific for a single epitope of the antigen. Monoclonal antibodies can be prepared according to methods described in the prior art (e.g. Köhler und Milstein, Nature, 256, 495-397, (1975); U.S. Pat. No. 4,376,110; Harlow und Lane, Antibodies: A Laboratory Manual, Cold Spring, Harbor Laboratory (1988); Ausubel et al., (eds), 1998, Current Protocols in Molecular Biology, John Wiley & Sons, New York). The disclosure of the mentioned documents is incorporated in total into the present description by reference.

Genetically engineered antibodies for use in the present invention may be produced according to methods as described in the afore-mentioned references. Briefly, antibody producing cells are cultured to a sufficient optical density, and total RNA is prepared by lysing the cells using guanidinium thiocyanate, acidification with sodium acetate, extraction with phenol, chloroform/isoamyl alcohol, precipitations with isopropanol and washing with ethanol. mRNA is typically isolated from the total RNA by chromatography over or batch absorption to oligo-dt-coupled resins (e.g. sepharose). The cDNA is prepared from the mRNA by reverse transcription. The thus obtained cDNA can be inserted into suitable vectors (derived from animals, fungi, bacteria or virus) directly or after genetic manipulation by “site directed mutagenesis” (leading to insertions, inversions, deletions or substitiutions of one or more bases pairs) and expressed in a corresponding host organism. Suitable vectors and host organisms are well known to the person skilled in the art. Vectors derived from bacteria or yeast such as pBR322, pUC18/19, pACYC184, Lambda oder yeast mu vectors may be mentioned as preferred examples. Such vectors are successfully used for cloning the corresponding genes and their expression in bacteria such as E. coli or yeast such as S. cerevisiae.

Antibodies for use in the present invention can belong to any one of the following classes of immunoglobulins: IgG, IgM, IgE, IgA, GILD and, where applicable, a sub-class of the afore-mentioned classes, e.g. the sub-classes of the IgG class. IgG and its sub-classes, such as IgG1, IgG2, IgG2a, IgG2b, IgG3 or IgGM, are preferred. IgG subtypes IgG1/k or IgG2b/k are especially preferred. A hybridoma clone which produces monoclonal antibodies for use in the present invention can be cultured in vitro, in situ oder in vivo. High titers of monoclonal antibodies are preferably produced in vivo or in situ.

Chimeric antibodies are species containing components of different origin (e.g. antibodies containing a variable region derived from a murine monoclonal antibody, and a constant region derived from a porcine immunoglobulin). Chimeric antibodies are employed in order to reduce the immunogenicity of the species when administered to the patient and to improve the production yield. For example, in comparison to hybridoma cell lines, murine monoclonal antibodies give higher yields. However, they lead to a higher immunogenicity in a non-murine, e.g. porcine, patient. Therefore, chimeric non-murine (in particular porcine)/murine antibodies are preferably used. Even more preferred is a monoclonal antibody in which the hypervariable complementarity defining regions (CDR) of a murine monoclonal antibody are combined with the further antibody regions of a non-murine, preferably porcine, antibody. Chimeric antibodies and methods for their production are described in the prior art (Cabilly et al., Proc. Natl. Sci. USA 81: 3273-3277 (1984); Morrison et al. Proc. Natl. Acad. Sci. USA 81:6851-6855 (1984); Boulianne et al. Nature 312 643-646 (1984); Cabilly et al., EP-A-125023; Neuberger et al., Nature 314: 268-270 (1985); Taniguchi et al., EP-A-171496; Morrion et al., EP-A-173494; Neuberger et al., WO 86/01533; Kudo et al., EP-A-184187; Sahagan et al., J. Immunol. 137: 1066-1074 (1986); Robinson et al., WO 87/02671; Liu et al., Proc. Natl. Acad. Sci. USA 84: 3439-3443 (1987); Sun et al., Proc. Natl. Acad. Sci. USA 84: 214218 (1987); Better et al., Science 240: 1041-1043 (1988) und Harlow und Lane, Antibodies: A Laboratory Manual, supra). The disclosure content of the cited documents is incorporated in the present description by reference.

According to the present invention, the term “antibody” comprises complete antibody molecules as well as fragments thereof being capable of binding to MSG1 or MSA1 or fragments, derivatives and analogues thereof as well as related proteins from other haemotrophic Mycoplasma species. Antibody fragments comprise any deleted or derivatised antibody moieties having one or two binding site(s) for the antigen, i.e. one or more epitopes of MSG1 or MSA1 or related molecules. Specific examples of such antibody fragments are Fv, Fab or F(ab′)₂ fragments or single strand fragments such as scFv. Double stranded fragments such as Fv, Fab or F(ab′)₂ are preferred. Fab und F(ab′)₂ fragments have no Fc fragment contained in intact antibodies. As a beneficial consequence, such fragments are transported faster in the circulatory system and show less non-specific tissue binding in comparison to complete antibody species. Such fragments may be produced from intact antibodies by proteolytic digestion using proteases such as papain (for the production of Fab fragments) or pepsin (for the production of F(ab′)₂ fragments), or chemical oxidation.

Preferably, antibody fragments or antibody constructs are produced through genetic manipulation of the corresponding antibody genes. Recombinant antibody constructs usually comprise single-chain Fv molecules (scFvs, ˜30 kDa in size), in which the V_{H} and V_{L} domains are tethered together via a polypeptide linker to improve expression and folding efficiency. In order to increase functional affinity (avidity) and to increase the size and thereby reduce the blood clearance rates, the monomeric scFv fragments can be complexed into dimers, trimers or larger aggregates using adhesive protein domains or peptide linkers. An example of such a construct of a bivalent scFv dimer is a 60 kDa diabody in which a short, e.g. five-residue, linker between V_{H}- and V_{L}-domains of each scFv prevents alignment of V-domains into a single Fv module and instead results in association of two scFv molecules. Diabodies have two functional antigen-binding sites. The linkers can also be reduced to less than three residues which prevents the formation of a diabody and instead directs three scFv molecules to associate into a trimer (90 kDa triabody) with three functional antigen-binding sites. Association of four scFvs into a tetravalent tetrabody is also possible. Further preferred antibody constructs for use in the present invention are dimers of scFv-CH₃ fusion proteins (80 kDa; so-called “minibodies”)

According to the present invention, one or more antisense nucleic acids, vectors (especially being capable of expressing the antisense nucleic acid(s)) and/or antibodies described herein are typically contained in a pharmaceutical composition containing the active ingredient(s) as described above as well as pharmaceutically acceptable excipients, additives and/or carriers (e.g. also solubilisers). Therefore, the present invention discloses a combination of the active ingredients as defined above and at least one pharmaceutically acceptable carrier, excipient and/or additive. Corresponding ways of formulating the pharmaceutical composition of the present invention are disclosed, e.g., in “Remington's Pharmaceutical Sciences” (Mack Pub. Co., Easton, Pa., 1980) which is part of the disclosure of the present invention. Examples of carriers for parenteral administration are, e.g., sterile water, sterile sodium chloride solutions, polyalkylene glycols, hydrogenated naphthalenes and, in particular biocompatible lactid polymers, lactid/glycolid copolymer or polyoxyethylene/polyoxypropylene copolymers. Such compositions according to the present invention are envisaged for the treatment of infections by M. suis as well as related haemotrophic Mycoplasma species. Moreover, compositions according to the present invention may contain fillers or substances such as lactose, mannitol, substances for covalently linking polymers such as, for example, polyethylene glycol to polypeptide, antibodies and derivatives or fragments thereof as disclosed in the present invention, for complexing with metal ions or for inclusion of materials into or on special preparations of polymer compounds such as, for example, polylactate, polyglycolic acid, hydrogel or onto liposomes, microemulsions, micells, unilamellar or multilamellar vesicles, erythrocyte fragments or spheroplasts. The particular embodiments of the compositions are chosen depending on the physical behaviour, for example with respect to the solubility, stability, bioavailability or degradability. A controlled or constant release of the active substance of the present invention in the composition includes formulations on the basis of lipophilic depots (e.g. fatty acids, waxes or oils). In the context of the present invention are also disclosed coatings of substances or compositions according to the present invention containing such substances, that is to say coatings with polymers (e.g. polyoxamers or polyoxamines). Furthermore, substances or compositions according to the present invention may comprise protective coatings such as protease inhibitors or permeability amplifying agents. The above optional ingredients may also be included in the vaccines of the present invention.

In principle, in the context of the present invention, all administration pathways known in the prior art for substances or compositions (vaccines, medicaments) according to the present invention are disclosed. Preferably, the administration of a medicament or vaccine for the treatment or prevention, respectively, of infections by M. suis or related species mentioned above is carried out via the parenteral, i.e., for example, subcutaneous, intramuscular or intravenous, oral or intranasal administration pathway. Vaccines containing polypeptides of the present invention are typically administered subcutaneously. In the case of genetic vaccines intramuscular injection is the preferred administration route. Typically, pharmaceutical compositions and vaccines according to the present invention will be solid, liquid or in the form of an aerosol (e.g. spray)—depending on the type of formulation.

Schedules for the treatment of and vaccination against infection by M. suis and related haemotrophic Mycoplasma species (e.g. M. wenyonii, M. haemofelis and M. haemocanis) are dependent on the individual to be treated, the severity of the infection and the type of molecule. A typical pharmaceutical/vaccine composition of the present invention contains 1 to 1000 μg of the active ingredient(s). The vaccine of the present invention is administered (via the routes as described above) one or more times to the subject to be immunised. Typically, the vaccine of the present invention is administered, e.g. as a 1:10 to 10:1, preferably 1:2 to 2:1, in particular 1:1 mixture with one or more adjuvants, in a primary immunisation which can be boosted by one or more further administrations which are typically separated by one or more weeks. A suitable schedule would be administration at day 0, 14, 21 and/or 28.

The pharmaceutical composition of the present invention may be administered once or more times daily over a time period effective for at least substantial reduction, preferably eradication of the pathogen in the infected individual.

Therefore, the above embodiments of the present invention, i.e. the polynucleotide, the antisense nucleic acid, the vector, the host cell, the polypeptide and/or the antibody are useful in therapy and/or prevention of infection by M. suis.

A further embodiment of the present invention relates to the production of the polypeptide as defined above, comprising the steps of:
(a) cultivating the host cell of the present invention and a suitable medium under conditions allowing the expression of the polypeptide; and
(b) recovering the polypeptide from the medium and/or host cells.

Preferably, the host cell to be cultivated according to step a) is produced by transforming a suitable host, e.g. by electroporation or chemical transfection of a suitable bacterium such as E. coli.

Step (b) of the method for the production of the polypeptide according to the present invention typically comprises conventional protein purification steps. In particular, host cell are commonly harvested (or removed from the medium containing the desired expression product) by centrifugation and may be disrupted by freeze/thawing cycles, sonification and/or application of high pressure. The cell lysate (in case the polypeptide is to be recovered from the cells) may be filtered and/or centrifuged. The cell lysate or the medium containing the polypeptide may be dialysed against suitable purification buffers which may be based on Tris, phosphate buffers etc. A further purification step may include a fractionated ammonium chloride precipitation. Further purification methods include with chromatographic fractionation steps by exchange chromatography, gel filtration chromatography and/or affinity chromatography using suitable resins, e.g. on the basis of dextran (e.g. sephadex), agarose (e.g. sepharose), polyacrylamide (e.g. sephacryl) or cellulose. Especially in case the polypeptide of interest is tagged suitably, e.g. with a His tag, a typical purification scheme includes an affinity chromatography, in particular metal chelate chromatography using Ni²⁺ or Zn²⁺ ions connected via a chelating group to a suitable resin. All chromatographic steps may be adapted to FPLC or HPLC equipment. In general, the person skilled in the art is readily able to set up and carry out a purification scheme depending on the source of or expression system used and depending on the nature (in particular amino acid sequence) of the protein of interest (cf., for example, Scopes, Protein Purification—Principles and Methods, 3^{rd} edition, Springer Verlag, Berlin, Germany, 1993; Deutscher (ed.), Guide to Protein Purification—Methods in Enzymology Edition, Vol. 182, Academic Press, San Diego, Calif., USA, 1990).

Based on the embodiments of the present invention, it is possible to establish methods, in particular diagnostics assays such as ELISA, immunoblot etc., for the detection of infections by haemotrophic Mycoplasma species, such as M. wenyonii in cattle, M. haemofelis in cats, M. haemocanis in dogs, and especially by M. suis in pigs, in all stages of a possible disease caused by such infectious particles. In particular, such detection methods are useful to detect carrier animals. For example, sera may be taken from clinically suspicious animals (serum peers) or from a representative number of animals within an animal herd such as a pig herd in order to carry out sera prevalence studies and herd diagnosis, respectively. Sera are investigated, for example, for their reactivity against the polypeptide of the present invention after a liquid dilution and in comparison to known positive and negative control sera.

Diagnostic assays of the present invention provide valuable means for the control of PE, since the infection may be re-indicated by detection and removal of infected carrier animals.

Therefore, generally speaking the present invention relates to diagnostic kits containing the polynucleotide and/or the antisense nucleic acid and/or the polypeptide and/or the antibody as defined above together with means for detection of said embodiments, i.e. the polynucleotide, antisense nucleic acid, polypeptide and/or antibody.

“Means for the detection” of the above-mentioned molecules are typically molecular markers that may be directly or indirectly attached with the polynucleotide, antisense nucleic acid, polypeptide and/or antibody. Such markers or labels may be selected from a variety of suitable compounds or chemical groups providing a directly or indirectly measurable signal such as characteristic light absorption, luminescence (in particular fluorescence), radioactivity etc. Specific examples are radioactive markers, fluorescence markers, dyes and members of specific binding pairs, such as biotin/streptavidin etc.

Preferably, the polynucleotide, antisense nucleic acid, polypeptide and/or antibody is/are coupled to a solid support such as membranes (for example nitrocellulose for nucleic acid molecules, or PVDF for peptides or polypeptides), resins, microbeads, culture dishes, wells of microtiter plates, microarrays etc.

A further embodiment of the diagnostic kit of the present invention contains a primer pair for amplifying a part, fragment or region of the sequence shown in FIG. 4A (SEQ ID NO: 1), preferably nucleotides 1397 to 2407 thereof, or 5A (SEQ ID NO: 3), preferably nucleotides 1792 to 3621 thereof, or related sequences having degrees of homology such that the primer pair is capable of successfully hybridising with such a related sequence, in particular sequences from haemotrophic Mycoplasma species other than M. suis. According to a preferred embodiment a diagnostic kit of the present invention comprises at least one oligonucleotide pair wherein one oligonucleotide (antisense oligonucleotide) comprises a sequence of at least 9, preferably 12, more preferred 15 nucleotides complementary to a sequence shown in FIG. 4A (SEQ ID NO: 1), more preferred nucleotides 1397 to 2407 thereof, or to a sequence shown in FIG. 5A (SEQ ID NO: 3), more preferred nucleotides 1792 to 3621 thereof, and the other oligonucleotide (sense oligonucleotide) comprises a sequence of at least 9, preferably 12, more preferred 15 nucleotides shown in FIG. 4A (SEQ ID NO: 1), more preferred nucleotides 1397 to 2407 thereof, or shown in FIG. 5A (SEQ ID NO: 3), more preferred nucleotides 1792 to 3621 thereof, respectively, wherein the 3′ most nucleotide in the sequence of the sense oligonucleotide is at least 20, preferably at least 50, more preferred at least 100 nucleotides upstream from the 3′ most nucleotide in the sequence of the antisense oligonucleotide.

The components of the diagnostic kit according to the present invention may be successfully used for the detection of M. suis and related species, in particular in the context of the detection of corresponding infections in susceptible animals, for example pigs, cattle, cats, dogs, horses, and human beings.

According to a preferred embodiment of the present invention a method for the detection of M. suis and related species comprises the steps of
(a) obtaining a sample suspected to contain M. suis (or a related species) or material derived therefrom;
(b) contacting the sample of step a) with at least one of the preferred embodiments disclosed herein, i.e. the polynucleotide, antisense nucleic acid, polypeptide and/or the antibody as defined above, under conditions allowing the binding of said polynucleotide, antisense nucleic acid, polypeptide and/or antibody to a component present in the sample/the material derived therefrom;
(c) performing one or more washing steps in order to remove any non-bound polynucleotide, antibody and/or antisense nucleic acid; and
(d) detecting the presence of said polynucleotide, antisense nucleic acid and/or antibody that has/have bound in step (b).

The above-defined method for the detection of haemotrophic Mycoplasma species such as M. suis can be adapted different forms depending on the specific molecule to be used for the detection of the infectious particle/specific component.

Thus, use of the polynucleotide and the antisense nucleic acid of the present invention typically relies on hybridisation with complementary sequences (or at least partially complementary sequences) present in the sample to be tested. Accordingly, when the polynucleotide and/or the antisense nucleic acid of the present invention are used, the present detection method or usually takes form of a Southern or Northern blot. Detailed experimental set-ups for such blotting techniques are well-known to the personal skilled in the art; cf. Sambrook et al., supra.

The polypeptide according to the present invention will be recognised by immunoglobulins, especially antibodies, present in the sample, which typically have been developed in an infected individual against M. suis or a related species. In turn, the antibody of the present invention will bind to a component in the sample by recognising the antigenic determined (epitope) the antibody is specific for.

When the polypeptide or the antibody according to the present invention is used for detection of components derived from M. suis (or from a related species) as disclosed herein, the detection method as defined above may take the form of a Western blot experiment but will typically be designed as an enzyme immunoassay, in particular an enzyme-linked immunosorbent assay (ELISA). Experimental set-ups and reagents (secondary antibodies, coupling chemistries etc.) are known to the skilled person (see, e.g., Anal. Methods Instrument. 1, 134-144 (1993), Coligan et al. (1991) Eds., Current Protocols in Immunology, Wiley, New York or Crowther, The ELISA Guidebook: Methods in molecular biology 49, Humana Press, Totowa N.Y., USA (2000)). Of course, other detection methods falling under the above definition can be envisioned. Typical examples other than enzyme immunoassays are assays of the radioimmunoassay type. Suitable techniques are disclosed in, e.g. Lefkovitz (Ed.), Immunology Methods Manual, Vol. 1-4, San Diego, Academic Press 1997 and Chard, An Introduction to Radioimmunoassay and Related Techniques, Amsterdam, Elsevier 1995.

The sample which is used for the detection method may be any specimen or sample which may contain M. suis or a related species. Preferred samples are derived from individuals (animals, humans) susceptible to infections by at least one of the pathogens in question. The sample may be any tissue (e.g. spleen) or body fluid derived from an individual susceptible to infection by M. suis or related species. Preferred body fluids are blood and blood products, especially serum, lymph fluid, cerebrospinal fluid, synovial fluid etc.).

A further preferred embodiment of a method for the detection of haemotrophic Mycoplasma species, preferably M. suis, or a corresponding diagnostic method relies on the amplification of a haemotrophic Mycoplasma-specific antigen encoding sequences as disclosed herein using at least one corresponding primer pair. Therefore, the present invention relates to a corresponding detection or diagnostic method comprising the steps of
(a) obtaining a sample suspected to contain M. suis (or a related species) or material derived therefrom (detailed examples are already given above);
(b) providing at least one primer pair specific for the sequences disclosed in FIG. 4A (SEQ ID NO: 1) or 5A (SEQ ID NO: 3) or a related sequence;
(c) performing a polymerase chain reaction (PCR) using the sample in step a) as template and the primer pair according to step b); and
(d) analysing amplification products produces in step c).

Preferably, the primer pair of the above method is defined according to the description of the diagnostic kit, supra. i.e. an oligonucleotide pair capable of serving as primers for PCR amplification of at least a part of the sequence disclosed in FIG. 4A (SEQ ID NO: 1), preferably nucleotides 1397 to 2407 thereof, or 5A (SEQ ID NO: 3), preferably nucleotides 1792 to 3621 thereof, or a related sequence. Of course, it is also possible to establish reversed transcriptase PCR (RT-PCR) methods on the basis of the sequences as disclosed herein or related sequences. As is known by a skilled person, RT-PCR methods may use only one sequence-specific primer whereas the other primer may be selected from unspecific primers such as a random hexamer primer or an oligo-dT primers. Corresponding PCR and RT-PCR kits and other products are commercially available from various manufacturers such as Stratagene (La Jolla, Calif., USA), BD Bioscience (Franklin Lakes, N.J. USA), Amersham Bioscience (Uppsala, Sweden) etc. PCR methods are known to the skilled person and specific experimental set-ups can be derived from various practical and theoretical references such as McPherson et al. (Eds.), PCR2, A Practical Approach, Oxford, IRL Press 1995; Rolfs et al., Methods in DNA Amplification, New York, Plenum Press 1994; Crit. Rev. Biochem. Mol. Bio. 26, 301-334 (1991).

An especially preferred embodiment of the amplification method for the detection of M. suis and related species or the diagnosis of an infection by such bacteria is provided by PCR methodology which enables the quantification of the produced PCR products either after amplification is completed (end point determination) or concomitantly during the amplification cycles (real-time PCR). Real-time PCR amplification protocols allow the quantification of the amount of the original template present in the test sample. General considerations and specific experimental set-ups of real-time PCR methods are reviewed, e.g. in Fenollar and Raoult, 2004 APMIS 112: 785-807. Therefore, based on the present invention, a real-time PCR assay is made available which is suited for the quantitative detection of M. suis or related species in blood as well as organ specimens derived from individuals susceptible to infection by M. suis or other haemotrophic Mycoplasma species. Using primer targets derived from unique encoding sequences disclosed herein which are specific for haemotrophic Mycoplasma species, the PCR assay of the present invention has the advantage of providing excellent specificity compared to assays based on ribosomal target sequences. In addition, the ease of standardisation and automation of PCR, especially real-time PCR techniques, as well as the effective prevention of contamination in such analytical set-ups allows the usage of the assay of the present invention in routine laboratories under comparative conditions. Therefore, a valuable comparison of the results obtained in different laboratories in different countries is made available.

The vector and the polypeptide according to the present invention are particularly useful for vaccination against infections by M. suis or related species. Therefore, the present invention also relates to vaccines comprising the inventive vector and/or the inventive polypeptide.

A vaccine containing at least one polypeptide according to the present invention thus comprises at least one antigenic determinant of the protein defined by the sequence disclosed in FIG. 4B (SEQ ID NO: 2) and/or FIG. 5B (SEQ ID NO: 4). The vaccine according to the present invention may also contain a polyprotein comprising multiple sequence fragments derived from the amino acid sequences shown in FIG. 4B (SEQ ID NO: 2) and/or FIG. 5B (SEQ ID NO: 4). Preferably, the vaccine according to the present invention contains one or more adjuvants and/or other immune stimulating agents. Suitable vaccines, in particular Freund's incomplete or complete adjuvant, are described above.

A further embodiment of the vaccine according to the present invention is represented by a genetic vaccine. The genetic vaccine according to the present invention comprises a vector as defined above, for example represented by an RNA- or DNA-based vector, suitably adapted to expression of the polypeptide according to the present invention. Thus, the genetic vaccine of the present invention preferably contains a vector which is suitable for expression of one or more antigenic determinants included in either or both of the sequences shown in FIG. 4B (SEQ ID NO: 2) and 5B (SEQ ID NO: 4). Of course, the vector contained in the genetic vaccine according to the present invention may contain a polygene coding for multiple epitopes contained in the sequences disclosed herein. Suitable genetic vaccines may be designed according to well-known principles which are reviewed, e.g. in Ivory et al. (2004) Genetic Vaccines and Therapy, 2, 17. Thus, the induction of T-cells by use of the genetic vaccine of the present invention provides a strategy to eliminate the pathogen (M. suis or related species) from infected individuals, especially animals such as pigs, cattle, cats and dogs.

A further embodiment of the present invention thus relates to a method for the prevention of an infection by M. suis or related species comprising the administration of an infective amount of the inventive vaccine as described above to an animal susceptible to infection by the corresponding pathogen.

As already disclosed above, the embodiments of the present invention are useful for therapy of infections by M. suis and related pathogens as well. Therefore, a pharmaceutical composition according to the present invention comprises a therapeutically active amount of the antisense nucleic acid and/or the vector and/or the antibody according to the present invention together with at least one pharmaceutically acceptable carrier, excipient and/or additive.

Accordingly, the antisense nucleic acid in the pharmaceutical composition of the present invention inhibits the expression of the polypeptide described herein thus elimination or at least controlling the pathogen. Furthermore, the vector capable of expressing the antisense nucleic acid as defined herein will generally function in the same way. The antibody according to the present invention is capable of binding to the immunodominant polypeptides derived from M. suis and related species such that the pathogen is significantly reduced or even eliminated after administration of the antibody.

The figures show:

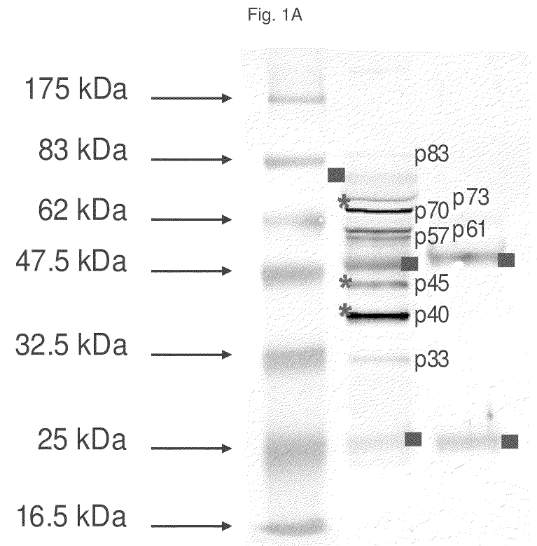

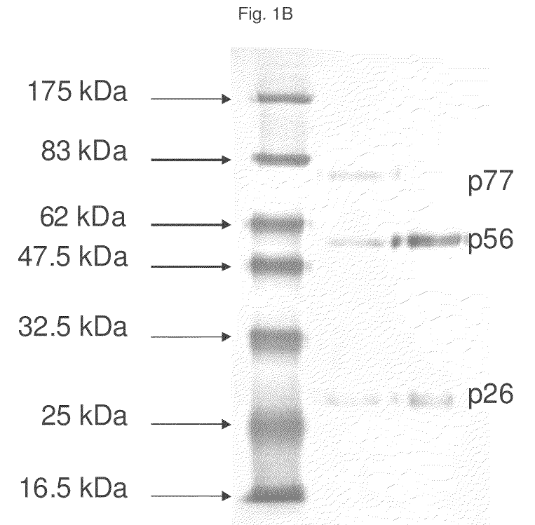

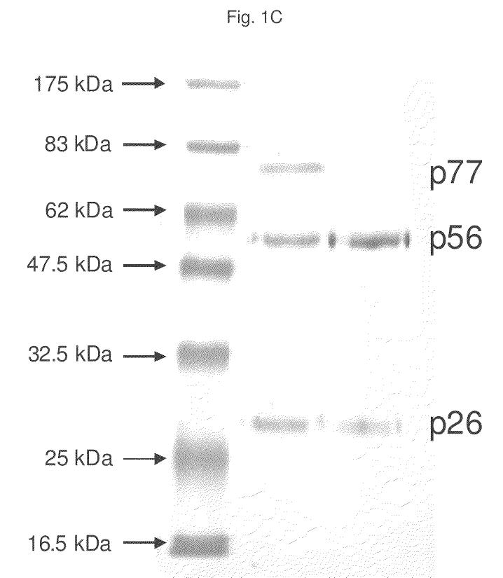

FIG. 1 shows photographs of one-dimensional Western blots of 10% Laemmli gels illustrating the detection of eight M. suis-specific antigens present in M. suis extracts obtained from whole blood of infected pigs. Panel (A) shows a blot incubated with serum obtained from M. suis-positive pig. Bands specifically reacting with M. suis-positive serum are indicated by their respective molecular weight. Immunodominant proteins (p40, p45 and p70) are marked with asterisks. Unspecific bands which were also detected by M. suis-negative serum and anti-pig Ig-conjugate are marked with rectangles (see panels (B) and (C), respectively: p26, p56 and p77). Panel (B) shows a corresponding control blot after incubation with M. suis-negative serum, and panel (C) shows a control blot after incubation with anti-pig Ig conjugate. Lanes in each blot from left to right: left lane: molecular weight marker; middle lane: M. suis extract from whole blood of infected pig; right lane: whole blood extract from non-infected pig.

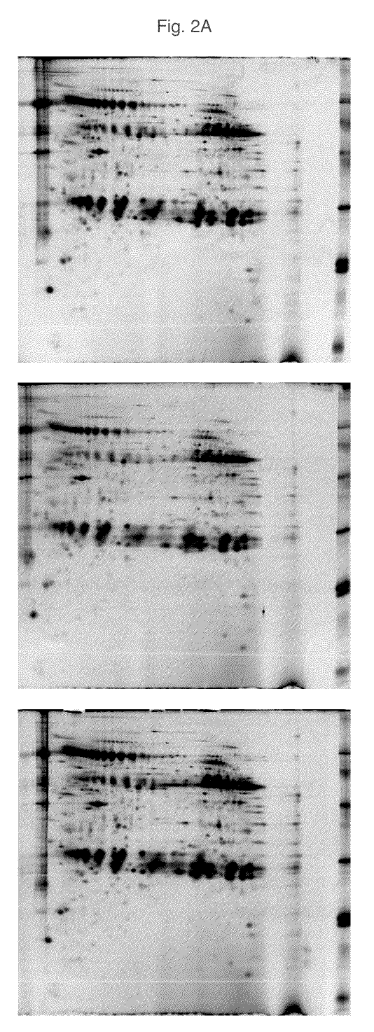

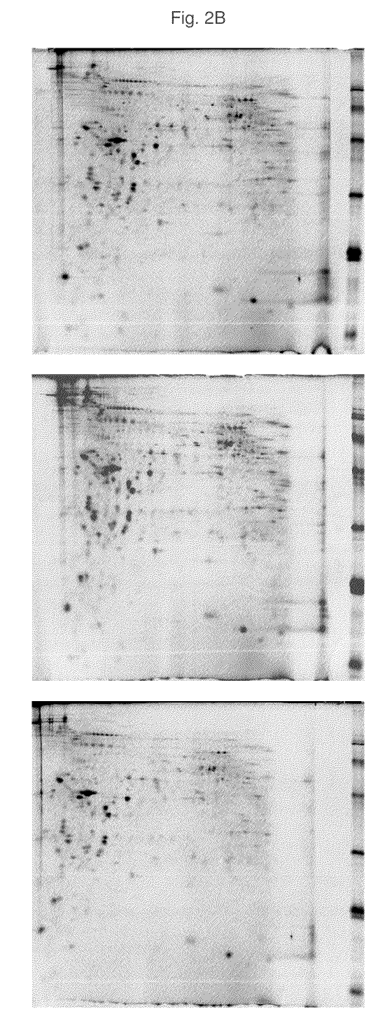

FIG. 2 shows photographs of triplicate two-dimensional SDS-PAGE analyses (isoelectric focussing/Laemmli) of (A) M. suis extracts obtained from the whole blood of infected pigs and (B) whole blood obtained from healthy control animals.

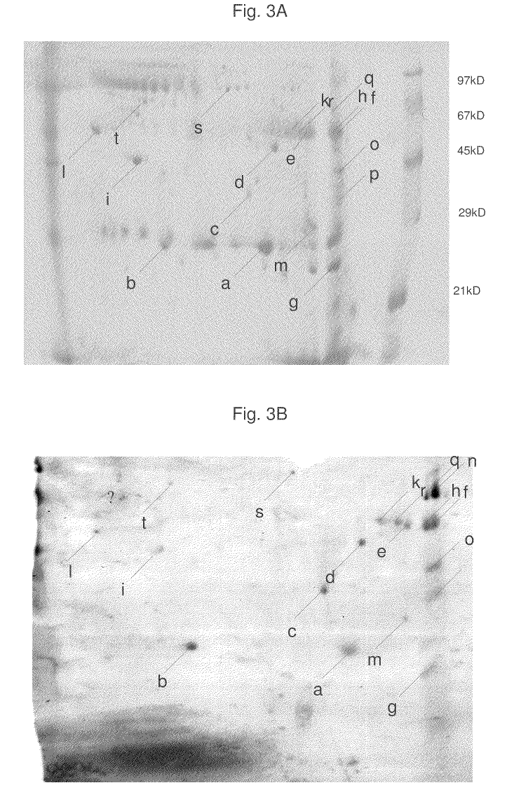

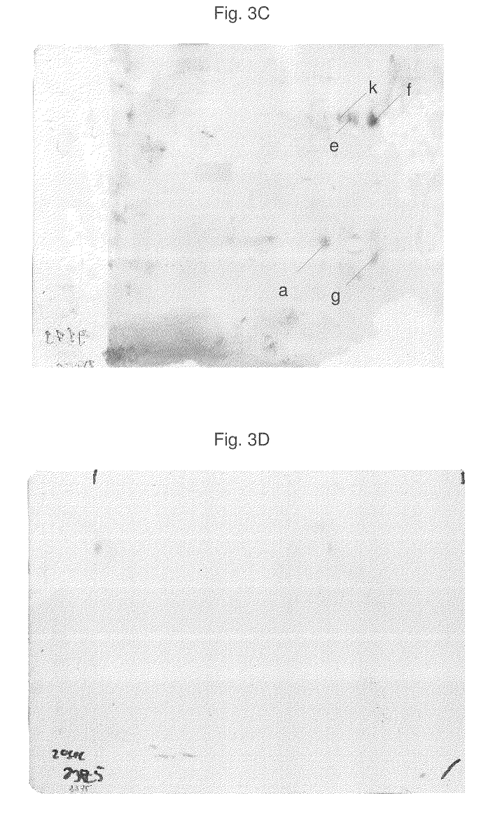

FIG. 3 shows photographs of two-dimensional Western blots demonstrating the identification of immunodominant M. suis polypeptides in sera of infected pigs. Panel (A) shows a Coomassie-stained 2-DE PVDF blot of an M. suis extract obtained from the whole blood of experimentally infected pigs. Panel (B) shows the same 2-D blot after incubation with serum from M. suis infected pigs. Panel (C) shows a control blot incubated with serum obtained from healthy control animals. Panel (D) shows a control blot incubated with secondary (anti-pig) antibody only. Spots reactive with the M. suis-positive serum are marked with letters. Spots a, e, f, g and k were not M. suis-specific, since they were recognised by the M. suis-negative serum as well (C). A spot apparently recognised by M. suis-positive serum but which could not be assigned properly is indicated with a question mark in (B).

FIG. 4 (A) shows the partial nucleotide sequence of the gene msg1. This genomic fragment comprises an open reading frame (ORF) of nucleotides 1397 to 2407 encoding an immunodominant protein (Mw about 40 kDa) of M. suis. Start and stop codons are marked in bold. (B) shows the deduced amino acid sequence of the protein (MSG1) derived from (A).

FIG. 5 (A) shows the partial nucleotide sequence of the gene msa1 comprising an ORF of nucleotides 1792 to 3627 coding for a further immunodominant protein (Mw about 70 kDa) of M. suis. Start and stop codons are marked in bold. (B) shows the deduced amino acid sequence of the protein (MSA1) derived from (A).

The present invention is further illustrated by the following non-limiting examples.

### EXAMPLES

### Identification of M. suis-Specific Antigens

Preparation of Mycoplasma suis Antigen

M. suis-infected whole blood was obtained from experimentally infected blood donor animals at maximum bacteriemia of acute clinical PE. 200 ml of peripheral whole blood were collected in 200 ml Alsever's solution at a 1:1 ratio. M. suis cells were purified as described previously (Hoeizle et al. (2003) Vet. Microbiol. 93: 185-196). In order to further purify M. suis cells from host cell components, the resulting M. suis pellet was resuspended in sterile PBS and was further purified from host cell components by centrifugation through 20% sodium diatrozoat meglumine and diatrozoat sodium (Urografin 76%, Schering, Berlin, Germany) at 25.000×g for 1 h at 4° C. (Allemann et al. (2001) J. Clin. Microbiol. 37: 1474-1479). The final pellet was resuspended in 1.0 ml PBS and stored at −80° C. until use (M. suis, (Ms) antigen). A negative control antigen was accordingly prepared from anti-coagulated blood of three non-infected animals which were confirmed as free of M. suis as described above.

### 1D-SDS-PAGE and Western Blot Analysis

Sodium dodecyl sulfate-polyacrylamide gel electrophoresis (SDS-PAGE) was performed according to standard procedures (Laemmli (1970) Nature 227: 680-685). Briefly, the Ms- and negative control antigen were boiled for 10 min in sample buffer containing 62.5 mM Tris (pH 6.8), 2.0% (wt/vol) sodium dodecyl sulfate, 25.0% Glycerol, 5.0% (vol/vol) β-mercaptoethanol, and 0.00125% bromphenol blue. Antigens were separated on 10.0% polyacrylamide gels (BioRad, Reinach, Switzerland, Miniprotean III; acrylamide/bisacrylamide ratio 37.5:1) with a protein loading concentration of 8.0 μg per track. Electrophoresis was performed under a constant voltage of 200 V until the dye front reached the bottom of the gel (˜40 min). Separated proteins were transferred onto nitrocellulose membranes (pore size 45 μm; BA85, Schleicher & Schuell, Riechen, Switzerland) using a semi-dry electrophoretic transfer cell (Trans Blot, BioRad), transfer buffer (25.0 mM Tris, 0.2 M glycine, 20.0% (vol/vol) Methanol) and a constant voltage of 10 V for 30 min. Membranes were blocked with 3.0% (wt/vol) nonfat dried milk in Tris-buffered saline (TBS, 0.01 M Tris, 0.15 M NaCl, pH 8.5) for 1 h. Thereafter, membranes were incubated for 2 h at 37° C. in the presence of sera from experimental piglets diluted 1:100 in blocking solution. A slot blot device (Multi-Screen apparatus, BioRad) was applied to analyze serial serum samples of eight experimentally infected pigs.

Membranes were washed twice with TBS for 10 min. Horseradish peroxidase-labeled goat anti-pig IgG (H+ L chain-specific, Sigma), goat anti-pig IgG (y chain specific, KPL-Bioreba, Reinach, Switzerland), and goat anti-pig IgM (μ chain specific, KPL) were used as secondary antibodies, respectively. All secondary antibodies were diluted 1:2000 in blocking solution. The blots were developed with H₂O₂ and 4-chloro-1-naphthol as the chromogenic reagents (BioRad). Enzymatic reactions were stopped by washing the blots in distilled water. Protein bands were sized with reference to molecular size marker lanes (prestained molecular size standard, 6.5 to 175 kDa, Bioconcept, Allschwil, Switzerland) using a computer-aided bio-image system (BioProfil 3.1, LTF, Wasserburg, Germany).

### Results

1-DE Western blotting using sera from experimentally infected animals revealed three main results:
i) an IgG immune response against M. suis-specific antigens is found during an infection,
ii) there are at least eight M. suis specific antigens (33 kDa, 40 kDa, 45 kDa, 57 kDa, 61 kDa, 70 kDa, 73 kDa, 83 kDa as determined by 10% SDS-PAGE according to Laemmli (FIG. 1A), and
iii) immune globulins (IgG, and IgM) are co-purified together with M. suis from the porcine blood (FIG. 1B, 1C).

The presence of co-purified immune globulins as a component of M. suis antigen preparations explained the fact that serological tests specific for M. suis are not available so far, since co-purified proteins complicate indirect serological assays such as ELISA which cannot differentiate between the proteins detected by the primary and those detected by the secondary antibodies.

Further purification of the M. suis antigen by removing the immunoglobulins allowed detailed studies of the immune response kinetics by immunoblot and ELISA.

Three M. suis-specific proteins are immunodominant (40 kDa, 45 kDa, 70 kDa; see FIG. 1A). All M. suis-infected animals showed a seroreactivity with at least one of the three immunodominant proteins during the second week of their infection at the latest and until the end of the experiment (18 weeks). Therefore, these three M. suis antigens are especially useful for serodiagnosis and vaccination.

Contrary to earlier studies, it is evident that the M. suis-specific humoral immune response shows no undulating course and basically follows the kinetics of a classical immune response against bacteria, i.e. initial presence of antibodies approx. 8-10 d post infection, ahead of first clinical symptoms, and a persistence of the M. suis-specific antibodies for months.

Partial Characterisation of M. suis-Specific Antigens by 2-DE Western Blotting/MALDI-TOF-MS

To further identify the nature and genetics of the immunoreactive M. suis proteins, 2-DE immunoblotting using patient sera pools was performed (immunoproteomics). The immunoreactive protein spots were further analysed by a peptide mass fingerprint (PMF) using matrix assisted laser desorption/ionisation-time of flight-mass spectrometry (MALDI-TOF-MS).

### 2-D Gel Electrophoresis and Western Blotting

The antigen samples (750 μl M. suis antigen and negative control antigen, respectively) were concentrated to a final volume of 200 μl using a spin column (Vivaspin, 10 kDa VS0101) with 4000×g at 10° C. Thereafter, the samples were diluted with 500 μl lysis buffer (7 M urea, 2 M thiourea, 4% CHAPS, 2% DTT, 1% [v/v] Pharmalyte pH 3-10). After shaking for 30 min at 20° C., the samples were centrifuged at 16000×g for 5 min at 20° C. The protein contents of aliquots of the clear supernatants was determined by the Bradford method α-test, Biorad), and samples were stored in aliquots at −80° C. until analyzed. 2D gels were loaded with 300 μg of total protein. In the first dimension (isoelectric focusing) the proteins were separated in 18 cm IPG (immobilized pH gradient) strips with a pH gradient ranging from pH 3 to 10 (Amersham Bioscience, Munich, Germany). Five gels of each sample were done with identical running conditions (30 kVh/IPG strip). After focusing to the steady state, the strips were loaded with SDS and equilibrated in DTT and Iodacetamide according to Görg (2000) Görg et al (2000) Electrophoresis 21: 1037-53. In the second dimension, the Laemmli buffer system was used (Laemmli (1970) Nature 227, 689). Proteins were separated with standard continuous 12% SDS gels which were run vertically in a Hoefer ISO-Salt chamber (AmershamBioscience) with 10 gels in parallel. In general between 1800 Vh and 2000 Vh were applied. The SDS PAGE was stopped, when the bromophenol blue front had disappeared from the gels.

After electrophoresis, the gels were removed from glass plates and stained with colloidal Coomassie (Roth, Heidelberg, Germany) according to the manufacturer's protocol. For western blotting a semidry blotting apparatus (Hoefer, AmershamBioscience) was used in which the unstained 2D gels were sandwiched with the PVDF membrane. The transfer buffer contained 50 mM Tris, 50 mM boric acid and 10% methanol (vol/vol); 1.5 mA/cm² were applied for 3 h.

The Coomassie stained 2D gels were used for protein identification by peptide mass fingerprinting, the Coomassie-stained PVDF blots were used for the immunological staining. In addition, Coomassie stained micropreparative gels were run with a 500 μg protein load per gel for the identification of low abundant spots.

Analysis of the 2D electropherograms of the M. suis antigen in comparison with the negative control antigen was done with the software Proteom Weaver (Definiens, ProteomWeaver Version 2.1.1).

### Protein Identification

Protein spots were identified by peptide mass fingerprinting (PMF)-MALDI-TOF analysis. Spots were cut out from the Coomassie-stained preparative gels, destained by washing thrice with 10 mM NH₄HCO₃, 30% acetonitrile (ACN). After digestion overnight in 5 μl trypsin buffer (25 ng/μl trypsin (Roche) dissolved in 10 mM NH₄HCO₃, pH 8) at 37° C., samples were kept in a sonication bath for 20 min at 25° C.

The supernatants were removed and concentrated using a Speedvac concentrator. For desalting the concentrated solution was processed through a C18 reversed phase ZipTip column (Millipore) and eluted with 0.1% trifluoroacetic acid (TFA) and 80% ACN. The eluted peptides were put on the target and co-crystallized with dihydroxybenzoeic acid (1 μl). MALDI-TOF analysis (Applied Biosystems Voyager STR) was performed in reflector mode in the peptide range from 700 to 4000 Daltons. The obtained spectra were matched with the NCBI database to identify the corresponding protein using the ProFound software (Genomic solution V. 2003).

### Results

Data of 6 of the above-mentioned M. suis antigens were obtained and are shown in Tab. 1 (see also FIG. 2).

TAB. 1
Results of MS/MS analyses of M. suis-specific antigens

| | | | | |
|---|---|---|---|---|
| | Molecular | | | |
| | weight (kDa)/ | | | |
| | isoelectric | Partial | Closest match in | NCBI |
| Spot | point (pl) | sequence | data library | acc. no. |

| | | | | |
|---|---|---|---|---|
| c_T | 72.4/4.8 | EELESNLGTIAK | class III heat shock protein | 15613570 |
| | | (SEQ ID NO: 5) | (chaperonin) [Bacillus | |
| | | | halodurans] | |
| d_T | 38.3/5.6 | SGKYDLDFKSPDDPSR | Eno 1 protein | 13278412 |
| | | (SEQ ID NO: 6) | [Mus musculus] | |
| I_T | 42.0/5.2 | VAPEEHPVLLTEAPLNPL | mutant beta actin | 28336 |
| | | (SEQ ID NO: 7) | [Homo sapiens] | |
| L_T | 51.02/6.0 | n.d.* | PROBABLE | 17545990 |
| | | | DIHYDROLIPOAMIDE | |
| | | | DEHYDROGENASE | |
| | | | (COMPONENT OF | |
| | | | PYRUVATE AND 2- | |
| | | | OXOGLUTARATE | |
| | | | DEHYDROGENASES | |
| | | | COMPLEXES) | |
| | | | OXIDOREDUCTASE | |
| | | | PROTEIN | |
| | | | [Ralstonia | |
| | | | solanacearum] | |
| o_T | 83.44/9.3 | n.d.* | DEAH-box protein involved | 6322772 |
| | | | in ribosome synthesis; | |
| | | | fragment, Dhr2p | |
| T_T | 53.6/5.6 | n.d.* | heat shock 70kDa protein | 24234686 |

* not determined

Cloning of Immunodominant M. suis-Specific Antigens

Experimentally infected pigs were used to construct a genomic library of M. suis. Screening of the library by hybridisation and shotgun sequencing revealed the full-length nucleotide sequences encoding the two immunodominant antigens (MSG1, 40 kDa; MSA1, 70 kDa). The nucleotide sequence of clone msg1 (SEQ ID NO: 1) is shown in FIG. 4A, which contains an ORF from nt 1397 to nt 2407. The deduced amino acid sequence of the protein MSG1 (SEQ ID NO: 2) is shown in FIG. 4B. The nucleotide sequence of clone msal (SEQ ID NO: 3) is shown in FIG. 5A, which contains an ORF from nt 1792 to nt 3621. The deduced amino acid sequence of the protein MSA1 (SEQ ID NO: 4) is shown in FIG. 5B.

The nucleotide sequences coding for the two proteins provide justification for the re-classification of Eperythrozoon suis to the genus Mycoplasma due to the codon usage found in these genes (see Benson et al. (2000) Nucleic Acids Res. 28: 15-18, Wheeler et al. (2000) Nucleic Acids Res. 28:10-14).

Recombinant Expression of Immunodominant M. suis-Specific Antigens

The two immunodominant antigens MSG1 and MSA1 were expressed recombinantly in E. coli after changing of the mycoplasmal codon usage to that of E. coli by synthetic gene engineering.

For recombinant expression, the coding sequences of the synthetic genes msg1 and msa1 were ligated into the pBADMycHis vector (Invitrogen, Netherlands). The ligation mixture was used to transform competent E. coli strain TOP10 for plasmid DNA isolation and E. coli strain LMG194 for protein expression. Transformants were selected from Luria Bertani (LB) agar plates supplemented with 100 μg/ml ampicillin. The correct orientation and nucleotide content of the introduced fragments were proofed by sequencing. Expression conditions were optimized for each plasmid construct. A volume of 200 ml of RM broth (Invitrogen) containing 100 μg/ml ampicillin were inoculated with 2 ml of a fresh overnight culture derived from a single colony of E. coli LMG194 transformants and grown at 37° C. to an optical density (OD) of 0.6 at 600 nm, equivalent to approximately 10⁸ cells/ml. 0.2% Arabinose was added to induce expression of MSG1 and MSA1 and cultures were incubated for further 1-4 h. Bacteria were harvested by centrifugation (5000×g, 15 min) and subjected to protein purification.

Purification of MSG1 and MSA1 from cytoplasmic protein aggregates of E. coli transformants was performed using Ni²⁺-NTA agarose (Qiagen). Bacterial pellets were resuspended in 20 ml PBS, and cells were lysed by ultrasonication on ice (35 W, 3×10 s). Insoluble material was removed by centrifugation (28 000×g, 30 min). The supernatant was mixed with 1 ml of Ni²⁺-NTA agarose. Tubes were incubated (120 min, 37° C.) with gentle agitation to allow maximum binding of His-tagged proteins. After centrifugation (3000×g, 10 min), the protein-laden Ni²⁺-NTA agarose was washed twice (3000×g, 10 min) with PBS containing 10 mM imidazole, and then MSG1 and MSA1 were eluted three times with 0.5 ml PBS containing 400 mM imidazole. The purified proteins were stored at −70° C.

SDS-PAGE and Western Immunoblotting of Recombinant M. suis-Specific Antigens

The immunogenicity of the recombinant proteins was tested by immunising rabbits and pigs. The utility of the recombinant proteins as antigens in serological assays could be confirmed in ELISA and Western blotting.

A volume of 500 ng of purified MSG1 and MSA1 as well as from negative controls (E. coli LMG194) were boiled for 10 min in 5× sample buffer [62.5 mM Tris pH 6.8, 10% (v/v) glycerol, 5% (v/v) 2-mercaptoethanol, 2.0% sodium dodecyl sulphate (SDS), 0.001% bromophenol blue] prior to electrophoresis through 2.4% polyacrylamide stacking and 10% polyacrylamide resolving gels at a constant voltage of 200 V using the Laemmli buffer system (Laemmli (1970) Nature 227: 680-685). Gels were stained with silver nitrate using the Silver Stain Plus Kit (BioRad, Reinach, Switzerland). For immunoblotting, proteins were transferred electrophoretically in 25 mM Tris, 192 mM glycine, 20% (v/v) methanol to a 0.45 μm-pore size nitrocellulose membrane (Schleicher & Schuell, Riechen, Switzerland). Membranes were blocked for 60 min at ambient temperature in Tris-buffered saline (TBS; 10 mM Tris, 150 mM NaCl, pH 7.5) containing 3% bovine serum albumin (BSA) (wt/vol). Membranes were incubated for 2 h at 37° C. with pig immune sera or rabbit immune sera (diluted 1:250 in TBS 3% BSA). Pre-immunization sera and antiserum raised against the E. coli LMG194 transformant containing the pBADMycHis plasmid without insert were used as controls. Membranes were washed twice with TBS for 10 min. Horseradish peroxidase-labelled rabbit anti-pig or goat anti-rabbit IgG (Sigma, diluted 1:1000 in TBS 3% BSA) were used as secondary antibodies. Antigen-antibody reactions were visualized with H₂O₂ and 4-chloro-1-naphthol as chromogenic reagents (BioRad). Enzymatic reactions were terminated by washing the blots in distilled water.

ELISA Using Recombinant M. suis-Specific Antigens

Microtitre plates (Microlon, Greiner, Nürtingen, Germany) were coated at 4° C. overnight with 100 μl per well of antigen (purified recombinant MSG1, MSA1 or E. coli LMG194-derived control antigen; f.c. 400 ng/ml) in carbonate-bicarbonate buffer (15.0 mM Na₂CO₃, 34.9 mM NaHCO₃, 3.1 mM NaN₃, pH 9.6). Phosphate-buffered saline (PBS; 136.9 mM NaCl, 1.46 mM KH₂PO₄, 8.1 mM Na₂HPO₄.2H₂O, 2.7 mM KCl, pH 7.4) containing 0.05% TWEEN® (polysorbate) 20 was used as the washing and incubation diluent. After coating, plates were washed three times by using an automated plate washer (Tecan, Maennedorf, Switzerland). Wells were blocked with 200 μl blocking buffer [PBS 0.05% TWEEN® (polysorbate) 20 with 1% (wt/vol) proteose peptone; Difco-Brunschwig, Basel, Switzerland]. The remaining washing and incubation steps were performed in 100-μl volumes per well and the wells were washed three times between the incubation steps. Incubations were performed for 1 h at ambient temperature starting with 15 min of constant agitation on a microtitre shaker. Plates were incubated with a 2-fold dilution range of each serum (1:200 to 1:102 400; rabbit, pig)). Each well then received a predetermined concentration of horseradish peroxidase-conjugated goat anti-rabbit IgG or rabbit anti-pig IgG (H+L chain specific, Sigma). Antigen-antibody reactions were visualized with 0.73 mM 2,2′-Azino-bis[3-ethylbenz-thiazolin-6-sulfonic acid] (ABTS) in 0.1 M citric-phosphate buffer pH 4.25 activated by the addition of 2 mM H₂O₂ immediately before use. Colour was allowed to developed for 20-30 min. OD values were recorded at 405 nm by a computer-assisted microplate reader (Tecan).

Diagnosis and Investigation of the Pathogenesis of M. suis Infections by Real-Time PCR

Based on the nucleotide sequences of the msg1 gene (encoding the approx. 40 kDa protein MSG1), it was possible to establish a real-time-PCR for the diagnosis and the investigation of the pathogenesis of M. suis infections.

For PCR amplification all blood samples (experimentally infected pigs, healthy control pigs) were prepared as follows: 200 μl-volumes of whole anti-coagulated blood were mixed with equal volumes of lysis buffer (10.0 mM Tris-HCl, pH 7.5, 5.0 mM MgCl₂, 0.32 M sucrose, 1% [v/v] Triton X-100) and centrifuged (8,000×g, 22° C., 60 s). The pellet was resuspended in 400 μl lysis buffer and again centrifuged. After repeating this step once, the pellet was resuspended in 400 μl PBS. DNA was extracted according to a standard protocol using phenol-chloroform-isoamyl alcohol (Sambrook and Russell (2001) Molecular cloning: a laboratory manual. 3rd edition, New York: Cold Spring Harbor Laboratory Press, Cold Spring Harbor) or with the MagNA Pure compact instrument (Roche Applied Science). The MagNA Pure Compact Nucleic Acid Isolation Kit I was used according to the manufacturer's instructions.

M. suis DNA was detected and quantified with the Light Cycler system (Roche Applied Science). The primers and hybridisation probes, defined in the msg1, were as follows: msg1f (sense), 5′-ACAACTAATGCACTAGCTCCTATC-3′ (SEQ ID NO: 8); and msg1r (antisense), 5′-GCTCCTGTAGTTGTAGGAATAATTGA-3′ (SEQ ID NO: 9).

The probes were: LC Red 640-5′-CAAG ACTCTCCTCACTCTGACCTAAGAAGAGC-Phosphate-3′ (SEQ ID NO: 10) and 5′-TTCACGCTTTCACTTCTGACCAAAGAC-3′-Fluorescein (SEQ ID NO: 11).

The size of the amplification product was 178 bp. Real-time PCR was carried out with the LightCycler Fast Start DNA Master^{PLUS} Hybridization Probes (Roche Applied Science). Extracted DNA (5 μl) was added to the 15 μl PCR mixture containing 4 μl Master Mix (5× conc.), 2 μl Primer-Probe Mix (10× conc. containing 0.5 μM end concentrations of each primer and 0.2 μM of each probe), and 9 μl water (PCR Grade). PCR conditions were as follows: initial denaturation of one cycle of 15 min at 95° C., followed by 40 cycles of 15 s at 95° C., 20 s at 60° C., and 10 s at 72° C. The reaction, data acquisition, and analysis were all done by using the Light Cycler instrument.

In summary, the above examples show:
detection of M. suis-specific antigens
detection of IgG immune response in M. suis infections
detection of three immunodominant M. suis-specific antigens which are valuable tools for diagnosis and vaccination
detection of the structure and function of immunodominant M. suis proteins
elucidation of the encoding genes of immunodominant M. suis proteins
recombinant expression of immunoreactive proteins derived from a member of the haemotrophic Mycoplasma species
recombinant production of test antigens for M. suis serology based on the antigens disclosed in the present invention can replace animal experiments and allows a high standardisation and uniformity of the test antigens
establishment of M. suis-specific recombinant serodiagnostic assays
establishment of M. suis-specific real-time-PCR assay

Embodiments of the present invention enable the diagnosis of and vaccination of infections with haemotrophic Mycoplasma species other than M. suis, e.g. M. wenyonii in cattle, M. haemofelis in cats, M. haemocanis in dogs. Establishment of pan-haemotrophic Mycoplasma-specific diagnostic assays will give more insight in the significance of such mycoplasmal microbes also in human beings.

What is claimed is:

1-23. (canceled)

24. A purified Mycoplasma suis polypeptide comprising an apparent molecular weight of about 40 kDa or 70 kDa in a continuous 12% polyacrylamide gel in 0.025 M Tris/0.192 M glycine/0.1% SDS aqueous solution and being reactive against serum from an M. suis positive animal.

25. A purified polypeptide comprising an amino acid sequence having at least 80% homology to an amino acid sequence set forth in SEQ ID NO:2 or SEQ ID NO:4.

26. A composition comprising the polypeptide of claim 24 and an adjuvant.

27. A purified polynucleotide that encodes the purified polypeptide of claim 24 or a fragment that encodes an amino acid sequence having at least 80% homology to at least 7 consecutive amino acids of SEQ ID NO:2 or SEQ ID NO:4.

28. An antisense nucleic acid directed against the purified polynucleotide of claim 27.

29. The purified polynucleotide of claim 27, wherein the polynucleotide comprises the nucleic acid sequence set forth in SEQ ID NO: 1 or SEQ ID NO:3.

30. An antisense nucleic acid directed against the purified polynucleotide of claim 29.

31. A purified antibody or a purified fragment of an antibody having a binding site, directed against the purified polypeptide of claim 24.

32. The purified antibody or fragment of an antibody of claim 31, wherein the purified antibody or fragment of an antibody is a polyclonal antibody, monoclonal antibody, Fv fragment, Fab fragment, F(ab′)₂ fragment, or scFv fragment.

33. A diagnostic kit comprising the purified polypeptide of claim 24 or a purified antibody directed against the purified polypeptide of claim 24, or a combination thereof.

34. The diagnostic kit of claim 33, wherein the purified polypeptide or purified antibody is coupled to a solid support.

35. A method for the detection of haemotrophic Mycoplasma species comprising:
(a) obtaining a sample suspected to contain haemotrophic Mycoplasma species or material derived therefrom;
(b) contacting the sample with the purified polypeptide of claim 24 or a purified antibody directed against the purified polypeptide of claim 24;
(c) performing one or more washing steps; and
(d) detecting binding between the purified polypeptide or antibody and a component of the sample.

36. The method of claim 35, wherein the detection is done by an ELISA, an immunoblot, a western blot, an enzyme immunoassay, or a radioimmunoassay.

37. The method of claim 35, wherein the purified polypeptide or the purified antibody comprise a marker or label.

38. The method of claim 35, wherein the purified polypeptide or the purified antibody is coupled to a solid support.

39. A method for the detection of haemotrophic Mycoplasma polynucleotides comprising:
(a) obtaining a sample suspected to contain haemotrophic Mycoplasma polynucleotides;
(b) providing at least one oligonucleotide pair capable of serving as primers for the amplification of at least a part of the polynucleotide sequence set forth in SEQ ID NO: 1 or SEQ ID NO:3 by polymerase chain reaction (PCR);
(c) performing a PCR using the sample as a template and the at least one oligonucleotide pair as primers;
(d) analyzing any amplification products produced by the PCR.

40. The method of claim 39, wherein the at least one oligonucleotide pair is capable of serving as primers for the amplification of at least a part of the sequence of nucleotides represented by nucleotides 1397 to 2407 of SEQ ID NO:1 or at least a part of the sequence of nucleotides represented by nucleotides 1792 to 3621 of SEQ ID NO:3.

41. The method of claim 39, wherein the amplification products are quantified.
